(19) United States
(12) Patent Application Publication
(10) Pub. No.: US 2012/0227132 A1
(43) Pub. Date: Sep. 6, 2012

(54) CELL NUMBER POLYNUCLEOTIDES AND POLYPEPTIDES AND METHOS OF USE THEREOF

(75) Inventors: Mei Guo, West Des Moines, IA (US); Carl R. Simmons, Des Moines, IA (US); Howard P. Hershey, Cumming, IA (US)

(73) Assignee: PIONEER HI BRED INTERNATIONAL INC, Johnston, IA (US)

(21) Appl. No.: 13/471,549

(22) Filed: May 15, 2012

Related U.S. Application Data
(63) Continuation of application No. 12/466,827, filed on May 15, 2009.
Application No. 12/466,827 is a continuation of application No. 11/153,071, filed on Jun. 15, 2005, now abandoned.
(60) Provisional application No. 60/583,340, filed on Jun. 28, 2004.

(51) Int. Cl. A01H 5/00; C12N 1/21; A01H 1/06; C12N 5/10; C12N 15/11; A01H 5/10; C12N 15/63; C12N 1/19

(52) U.S. Cl. 800/278; 435/320.1; 435/252.33; 435/252.31; 435/252.3; 435/254.21; 435/254.23; 435/348; 435/358; 435/352; 435/369; 435/419; 435/415; 435/412; 435/414; 536/23.1; 800/298; 800/320.1; 800/312; 800/320; 800/306; 800/320.3; 800/314; 800/320.2; 800/322

(57) ABSTRACT

The present invention provides polynucleotides and related polypeptides of the protein CNR. The invention provides genomic sequence for the CNR gene. CNR is responsible for controlling cell number.

### CROSS REFERENCE

This utility application is a continuation of U.S. patent application Ser. No. 12/466,827 filed May 15, 2009 which is a continuation of U.S. patent application Ser. No. 11/153,071 originally filed Jun. 15, 2005 and continued Jul. 15, 2008, which claims the benefit U.S. Provisional Application Ser. No. 60/583,340, filed Jun. 28, 2004, all of which are incorporated herein by reference.

### FIELD OF THE INVENTION

The invention relates generally to the field of molecular biology.

### BACKGROUND OF THE INVENTION

The domestication of many plants has correlated with dramatic increases in yield. Most phenotypic variation occurring in natural populations is continuous and is effected by multiple gene influences. The identification of specific genes responsible for the dramatic differences in yield, in domesticated plants, has become an important focus of agricultural research.

One group of genes effecting yield are the cell number regulator genes, or CNR genes. One quantitative trait locus (QTL), fw2.2, containing the specific gene family, was found to be associated with decreases of up to 30% in fruit size in tomatoes. The suggestion is that fw2.2 may be a negative regulator of cell division. (Frary, et al., (2000) Science 289:85-88) The plants producing the fruit did not differ significantly in the total fruit weight, or harvest index (fruit yield divided by plant weight). Alterations in the fruit size, as imparted by the fw2.2 alleles appeared to be due to changes in regulation rather than in the sequence and structure of the encoded protein. The cause of the QTL effect in the tomato plants was a single gene controlling carpel cell number that is expressed early in the floral development. Further research indicated that the primary effect of fw2.2 is in controlling ovary and fruit size, and that other associated phenotypic effects are secondary. (Nesbitt, et al., (2001) Plant Physiology 127:575-583). The fw2.2 genes were found to change plant morphology by modifying sink-source relationships at the whole plant level, causing alteration of inflorescence number, fruit number, and fruit and flower abortion rates. The genes have the potential to alter the competition for photosynthate among fruit, thereby having a significant impact on the size of fruit at maturity.

Heterosis is an important mechanism whereby many crop plants are enhanced in yield and performance. It is characterized by an increase in plant growth and vigor that gives rise to enhanced yield. This heterotic gain usually occurs in hybrid offspring of select parent lines that may otherwise be middling in yield performance. These parent lines are often inbred and relatively genetically homogenous lines. While heterosis is used in several crop plant production systems, its application to maize is most widely known. It is used in the majority of maize production systems in the developed world. It is the underpinning to the hybrid seed corn industry.

The full mechanism of heterosis is still being investigated. While not intending to be bound by any one theory, proposed heterosis mechanisms are listed here. There is a hypothesis which envisions a complementation of multiple gene alleles, whether by protein function, by gene expression or otherwise. However other non-exclusive theories envision a smaller set of key genes, especially those whose alteration in function might result in changes in plant vigor.

### SUMMARY OF THE INVENTION

The present invention provides polynucleotides, related polypeptides and all conservatively modified variants of the present CNR sequences. The invention provides sequences for the CNR genes.

Genes that regulate cell number in plants have the potential to be involved in heterosis. Cell-number regulating genes could be judiciously manipulated to achieve a heterotic phenotype. Heterotic plants are usually more robust and of larger stature. Few studies have investigated the source of this greater size. One key observation made by maize biologist T. A. Kiesselbach in 1922 revealed that in maize heterosis is primarily due to increases in cell number not cell size. (See, Kiesselbach, (1922) Corn Investigations Bulletin of the Agricultural Experiment Station of Nebraska. Research Bulletin No. 20. The University of Nebraska. Lincoln, Nebr. U.S.A. Histological Effects of Inbreeding, pages 96-102.) Specifically Kiesselbach states, “These data suggest that 10.6 percent of the increased size due to crossing results from an increase in cell size and 89.4 percent of it from increased numbers”.

The tomato gene Fw2.2 can be defined as a negative regulator of cell number in tomato carpels. When the Fw2.2 gene expression is diminished, larger tomato fruit are produced. The fruit have been analyzed microscopically and show the larger fruit to be due to increases in cell number not cell size. It is consistent with the effect of heterosis, thus maize orthologues to the tomato gene Fw2.2 are desirable.

The present invention identifies maize genes encoding proteins related to the gene family containing tomato Fw2.2 gene. These genes could be used to produce the heterotic phenotype. Such a phenotype would exhibit diminishment of the negative regulation of cell number leading to a plant, or part(s) of a plant, that are larger (larger cell number) and more vigorous, ultimately leading to enhanced crop plant yield.

Each of the CNR (cell number regulator) genes, and any other genes of this gene family, may be useful for enhancing crop yield. One of the CNR genes, ZmCNR02 is of particular interest in that (a) it is similar in amino acid identity to the tomato Fw2.2 proteins, and (b) its natural expression in multiple tissues is consistent with negative regulation of cell number and that its expression goes up in mature or maturing tissues, which would be shutting down new cell production.

The CNR genes are negative regulators of cell number and their decreased expression causes increased cell number, enhanced vigor, and heterotic phenotype. If the ZmCNR02 gene were to be downregulated (by knockout, mutation, homologous recombination, antisense, microRNAs or otherwise), then increased cell numbers would result. Provided that this downregulation does not deleteriously affect normal balance in development, a larger-sized, more vigorous plant may result. The downregulation may be partial or complete, increasing cell number without imbalance in development, or the knockout or diminished expression in particular tissues (such as endosperm) could result in larger organs, by virtue of extending their period of cell division. These approaches could be used in addition to conventional heterosis, giving an added yield enhancement.

The CNR02 gene shows expression in diverse tissues, and may control cell number throughout the plant. General heterosis tends to make the whole plant more vigorous, not simply one or a set of tissues. Accordingly, the above strategies requiring promoters to diminish the gene expression (esp. of ZmCNR02) may be tried using promoters with a broad developmental expression pattern. These may be among the so-called constitutive promoters, one example being the maize ubiquitin promoter. Promoters could also be used to focus expression in one or several tissues (if desired enhanced cell number is deemed to be sufficient within a limited spectrum of the plant development). Such enhanced tissues of interest include for example, roots (enhance root development by diminishing gene or gene expression there), embryos (larger embryos, including effecting higher oil content of the whole seed), seedling (seedling vigor, enhanced mesocotyl size and extension to emerge more successfully from the soil), silks (enhanced silk emergence, including during droughted conditions, or to nick or synchronize with pollen donation), stalks (to increase the girth leading to greater stalk strength) and other plant tissues of interest.

CNR gene function has a relationship to auxin and cadmium in growing tissue. While not being bound by any particular theory, a possible molecular/physiological mode of action of the CNR-type genes affecting plant cell number is presented. This hypothesis is not represented in the public literature and reconciles information that would otherwise appear confusing or contradictory.

The connections between CNR genes, growth, auxin action and cadmium, lead to the following theorized mode of action.

The CNR genes affect cell number through auxin action, a well known plant hormone that affects plant cell growth, in some instances through cell number. Cadmium in a general metal binding capacity, also has a role in auxin action, where a substitution by cadmium occurs where there would otherwise be a zinc ion that is involved in auxin binding or action. The CNR gene expression may increase as tissues mature in order to bind to auxin and check the continued growth of the tissue. In this way the CNR genes may bind auxin to help to stop its growth-promoting action.

One reason that CNR genes have not been found to be auxin binding proteins to date may be in part because they are membrane bound (and thus less likely to be isolated). Another reason may be because their expression may be higher in maturing tissues, an area where modes of auxin action have been investigated to a lesser degree. Plants over-expressing CNR genes may be auxin resistant and/or cadmium resistant. The auxin resistance and cadmium resistance may be in conflict as cadmium replaces zinc and undermines the auxin-binding activity. The auxin resistance could mean resistance to auxin-related herbicides. The cadmium resistance may be efficacious in certain settings for resistance of plants to toxic metals. Reductions in CNR expression or activity would result in auxin sensitivity and/or cadmium sensitivity. The former may result in increased sensitivity to auxin-related growth, which is the mode of action of auxin-related herbicides. When CNR genes affect auxin action, they could affect both cell number and cell size, as auxin does. So, while CNR genes affecting cell size is the result of the Fw2.2 gene, and is expected to be the general effect of modulating these genes' expression, one would not rule out a possible accompanying effect on cell size.

The fw2 tomato gene family has at least 12 members related to it in maize, designated as ZmCNR1-12 (see, Table 1). These genes may be used to control cell number in the maize plants. Potential uses include controlling the size of whole plants, or specific organs within the crop plant. They genes may also be used to control seed and fruit size. Proper control of the gene can result in whole organs being enhanced in size, or reduced or eliminated altogether. Either outcome would be agronomically advantageous. While the gene may generally function as a negative regulator of cell number and thus organ size in its normal wild type function, it is recognized that this function could be altered by judicious manipulation of the level or timing of the expression of the gene, or by altering or disrupting the coding region of the gene. In that instance, the function of the gene product, a protein, is altered. Potential outcomes include but are not limited to: increased leaf size, increased root size, increased ear size, increased seed size which could include increased endosperm size, alteration in the relative size of embryos and endosperm which in turn would effect changes in the relative levels of protein, oil and starch in the seeds and elimination of tassels, or at least functional pollen bearing tassels.

RT-PCR data shows that ZmCNR02 expressed highly in ovules prior to fertilization, but declined thereafter. This is consistent with the gene being involved in negative cell number regulation. This observation suggests ways to further exploit this and related genes for crop improvement. One area is in fertilization independent seed production. The phenomenon of negative cell number regulation (by diminishing CNR expression and/or function) in the ovules could result in seed or seed-like formation in the absence of fertilization. Such seeds may be viable to germinate in the next generation, or they could be useful for seed production for food, fuel and the other normal consumption roles of seeds. There are a number of possible novel uses and virtues for fertilization independent seed formation that could affect the agriculture industry. Haploids are increasingly being used for parent line production in hybrid crop (maize) production. A potential parent plant can be identified as a haploid and then doubled to a diploid to achieve a homozygous parent line without the need for inbreeding.

A further embodiment of the invention includes methods for controlling the CNR or the function of related proteins, and for reducing the activity of these proteins in order to express a modified nonfunctional version of such proteins. This may disrupt function of the intact natural versions of the genes by blocking or competing for sites of action. The promoter of the CNR gene could be used to direct expression to ovules. Applications include novel means for regulating the CNR or related genes in the ovule as mentioned above, and in other areas where control of gene expression in the ovule is needed. The CNR genes are expressed in many tissues besides the ovule and that expression is increased where tissues are maturing or mature.

One application for the CNR and related genes includes altering the formation of tissue by controlling cell division. For example, controlling functional tassel formation is an important aspect of hybrid maize production. The CNR gene can be operably linked to a tassel-, anther- or tapetum-specific promoter, thereby controlling the development of those tissues, leading to an alteration of male sterility.

TABLE 1

| SEQUENCE ID NUMBER | IDENTITY |
|---|---|
| SEQ ID NO: 1 | ZmCNR 1 polynucleotide |
| SEQ ID NO: 2 | ZmCNR 1 polypeptide |
| SEQ ID NO: 3 | ZmCNR 2 polynucleotide |
| SEQ ID NO: 4 | ZmCNR 2 polypeptide |
| SEQ ID NO: 5 | ZmCNR 3 polynucleotide |
| SEQ ID NO: 6 | ZmCNR 3 polypeptide |
| SEQ ID NO: 7 | ZmCNR 4 polynucleotide |
| SEQ ID NO: 8 | ZmCNR 4 polypeptide |
| SEQ ID NO: 9 | ZmCNR 5 polynucleotide |
| SEQ ID NO: 10 | ZmCNR 5 polypeptide |
| SEQ ID NO: 11 | ZmCNR 6 polynucleotide |
| SEQ ID NO: 12 | ZmCNR 6 polypeptide |
| SEQ ID NO: 13 | ZmCNR 7 polynucleotide |
| SEQ ID NO: 14 | ZmCNR 7 polypeptide |
| SEQ ID NO: 15 | ZmCNR 8 polynucleotide |
| SEQ ID NO: 16 | ZmCNR 8 polypeptide |
| SEQ ID NO: 17 | ZmCNR 9 polynucleotide |
| SEQ ID NO: 18 | ZmCNR 9 polypeptide |
| SEQ ID NO: 19 | ZmCNR 10 polynucleotide |
| SEQ ID NO: 20 | ZmCNR 10 polypeptide |
| SEQ ID NO: 21 | ZmCNR 11 polynucleotide |
| SEQ ID NO: 22 | ZmCNR 11 polypeptide |
| SEQ ID NO: 23 | ZmCNR 12 polynucleotide |
| SEQ ID NO: 24 | ZmCNR 12 polypeptide |
| SEQ ID NO: 25 | ZmCNR 2 polypeptide |
| | full length with ORF identification |
| SEQ ID NO: 26 | ZmCNR 2 open reading frame |
| SEQ ID NO: 27 | ZmCNR 2 polypeptide |
| | translation of SEQ ID NO: 26 |
| SEQ ID NO: 28 | ZmCNR 1 Promoter |
| SEQ ID NO: 29 | ZmCNR 2 Promoter |
| SEQ ID NO: 30 | ZmCNR 4 Promoter |
| SEQ ID NO: 31 | ZmCNR 6 Promoter |
| SEQ ID NO: 32 | ZmCNR 7 Promoter |
| SEQ ID NO: 33 | ZmCNR 9 Promoter |
| SEQ ID NO: 34 | ZmCNR 11 Promoter |
| SEQ ID NO: 35 | ZmCNR 12 Promoter |
| SEQ ID NO: 36 | ZmCNR 1 MPSS preferred Tag |
| SEQ ID NO: 37 | ZmCNR 2 MPSS preferred Tag |
| SEQ ID NO: 38 | ZmCNR 3 MPSS preferred Tag |
| SEQ ID NO: 39 | ZmCNR 5 MPSS preferred Tag |
| SEQ ID NO: 40 | ZmCNR 6 MPSS preferred Tag |
| SEQ ID NO: 41 | ZmCNR 7 and ZmCNR 9 MPSS |
| | preferred Tag |
| SEQ ID NO: 42 | ZmCNR 8 MPSS preferred Tag |
| SEQ ID NO: 43 | ZmCNR 10 MPSS preferred Tag |
| SEQ ID NO: 44 | ORF translation of homologue to |
| | Lycopersicon esculentum fw2.2 |
| SEQ ID NO: 45 | Lycopersicon esculentum fw2.2 |

Therefore, in one aspect, the present invention relates to an isolated nucleic acid comprising an isolated polynucleotide sequence encoding a CNR protein. One embodiment of the invention is an isolated polynucleotide comprising a nucleotide sequence selected from the group consisting of: (a) the nucleotide sequence comprising SEQ ID NO: 1, 3, 5, 7, 9, 11, 13, 15, 17, 19, 21 or 23; (b) the nucleotide sequence encoding an amino acid sequence comprising SEQ ID NO: 2, 4, 6, 8, 10, 12, 14, 16, 18, 20, 22 or 24 and (c) the nucleotide sequence comprising at least 70% sequence identity to SEQ ID NO: 1, 3, 5, 7, 9, 11, 13, 15, 17, 19, 21 or 23, wherein said polynucleotide encodes a polypeptide having cell number regulator activity.

Compositions of the invention include an isolated polypeptide comprising an amino acid sequence selected from the group consisting of: (a) the amino acid sequence comprising SEQ ID NO: 2, 4, 6, 8, 10, 12, 14, 16, 18, 20, 22 or 24 and (b) the amino acid sequence comprising at least 70% sequence identity to SEQ ID NO: 2, 4, 6, 8, 10, 12, 14, 16, 18, 20, 22 or 24, wherein said polypeptide has cell number regulatory activity.

In another aspect, the present invention relates to a recombinant expression cassette comprising a nucleic acid as described. Additionally, the present invention relates to a vector containing the recombinant expression cassette. Further, the vector containing the recombinant expression cassette can facilitate the transcription and translation of the nucleic acid in a host cell. The present invention also relates to the host cells able to express the polynucleotide of the present invention. A number of host cells could be used, such as but not limited to, microbial, mammalian, plant or insect.

In yet another embodiment, the present invention is directed to a transgenic plant or plant cells, containing the nucleic acids of the present invention. Preferred plants containing the polynucleotides of the present invention include but are not limited to maize, soybean, sunflower, sorghum, canola, wheat, alfalfa, cotton, rice, barley, tomato and millet. In another embodiment, the transgenic plant is a maize plant or plant cells. Another embodiment is the transgenic seeds from the transgenic plant. Another embodiment of the invention includes plants comprising a CNR polypeptide of the invention operably linked to a promoter that drives expression in the plant. The plants of the invention can have altered cell number as compared to a control plant. In some plants, the cell number is altered in a vegetative tissue, a reproductive tissue or a vegetative tissue and a reproductive tissue. Plants of the invention can have at least one of the following phenotypes including but not limited to: increased leaf size, increased ear size, increased seed size, increased endosperm size, alterations in the relative size of embryos and endosperms leading to changes in the relative levels of protein, oil, and/or starch in the seeds, absence of tassels, absence of functional pollen bearing tassels or increased plant size.

Another embodiment of the invention would be plants that have been genetically modified at a genomic locus, wherein the genomic locus encodes a CNR polypeptide of the invention.

Methods for increasing the activity of a CNR polypeptide in a plant are provided. The method can comprise introducing into the plant a CNR polynucleotide of the invention. Providing the polypeptide can decrease the number of cells in plant tissue, modulating the tissue growth and size.

Methods for reducing or eliminating the level of a CNR polypeptide in the plant are provided. The level or activity of the polypeptide could also be reduced or eliminated in specific tissues, causing increased cell number in said tissues. Reducing the level and/or activity of the CNR polypeptide increases the number of cells produced in the associated tissue.

Methods and compositions for regulating gene expression in a plant are also provided. Polynucleotides comprising promoter sequences are provided (see, Table 1). Compositions include isolated polynucleotides comprising a nucleotide sequence selected from the group consisting of: (a) the nucleotide sequence comprising SEQ ID NO: 28, 29, 30, 31, 32, 33, 34 or 35; and (b) the nucleotide sequence comprising at least 70% sequence identity to SEQ ID NO: 28, 29, 30, 31, 32, 33, 34 or 35. Compositions further include plants and seed having a DNA construct comprising a nucleotide sequence of interest operably linked to a promoter of the current invention. In specific embodiments, the DNA construct is stably integrated into the genome of the plant. The method comprises introducing into a plant a nucleotide sequence of interest operably linked to a promoter of the invention.

### BRIEF DESCRIPTION OF THE FIGURES

FIG. 1. Clustal Dendogram of Tomato Fw2-2 (SEQ ID NO: 45) with 12 Maize Gene Translations (SEQ ID NOS: 2, 4, 6, 8, 10, 12, 14, 16, 18, 20, 22 and 24).

FIG. 2. Alignment of Tomato Fw2-2 (SEQ ID NO: 45) with 12 Maize Gene Translations (SEQ ID NOS: 2, 4, 6, 8, 10, 12, 14, 16, 18, 20, 22 and 24).

FIG. 3. Endosperm Development. The pattern of ZmCNR02 gene expression as revealed by MPSS data reveals that the gene expression is very low in the early stages of endosperm development (in early days after pollination—DAP), but that as the endosperm matures (higher DAP), the expression of CNR02 increases. Thus this pattern of expression in endosperm is consistent with a role of CNR02 in negatively regulating cell number.

FIG. 4A. Embryo Development. The seed embryo development is scored in terms of days after pollination (DAP). The pattern of ZmCNR02 expression rises towards the end of embryo development after 30 DAP, with the highest expression at 45 DAP. This corresponds to the period of completion of cell number growth, this pattern of expression is consistent with a role for ZmCNR02 as a negative cell number regulator.

FIG. 4B. RT-PCT analysis of ZmCNR02 expression in different maize tissues. Thirty-five cycles of RT-PCR was performed with different maize tissues, including endosperm (14 DAP), shoot apical meristem, pericarp, seedling, root, brace root, mature and immature leaf, immature ear, immature tassel, node and ovule. Consistent with the Lynx MPSS profiling data, the expression of this gene is detected mostly in the tissue where there is little growth activity, such as mature leaf. Interestingly, a very high expression is detected in the ovule tissue. The ovule (pre-fertilization) has no cell division activity and is at a rest stage. ZmCNR02 is expressed at a very high level in the ovule, comparable to the level in the mature leaf tissue. However, immediately after fertilization when active cell division begins, the ZmCNR02 expression is dramatically reduced to a minimal level (demonstrated by the early embryo and endosperm development illustrated in FIGS. 3 and 4A).

FIG. 5. Leaf Development. Several samples were assayed in relation to developing maize leaves. The basal region of immature leaves, the region of most active cell division, showed no ZmCNR02 expression. The distal expanding and expanded portions of the same immature leaves showed a small but noticeable ZmCNR02 expression. A series of whole leaves from young plants (V2) to middle stage leaves (V7-V8) to mature leaves, showed progressively higher ZmCNR02 expression. This expression pattern is consistent with ZmCNR02 being related to negative control of cell number; its expression is highest in leaf stages that are undergoing little cell division.

FIG. 6. Carpels, Silk Development and Pollen. The silks, ovary walls and pericarp are analogous to the dicot flower carpel. ZmCNR02 expression is detected in the latter two. The ZmCNR02 expression is in the maize ‘carpels’ by virtue of the silk and pericarp expression. The pericarp samples assayed are fairly late in development and are compromised by remaining endosperm tissue. The silk tissues are fairly easy to gather and assay for gene expression. In the young growing silks (those still attached to the ovaries) the expression of ZmCNR02 expression is not detected. Then moving through a series of pre-emergent to post emergent silks, and thence through a post pollination series, the expression of ZmCNR02 increases. For comparison the pollen sample is offered indicating that the increase of ZmCNR02 expression is not derived from the pollen landing on the silks. As silks mature, and especially after they are pollinated, the cell division slows and stops. The pattern of ZmCNR02 expression in silks (a carpel tissue) is consistent with a negative cell number regulator.

FIG. 7. Root and Root Meristems. A comparison of whole roots (with meristems) to root tips (meristem enriched), shows that ZmCNR02 expression is higher in whole roots than root tips. The ZmCNR02 expression having higher expression in areas of the root not actively dividing, and the expression pattern in roots is consistent with as a negative regulator of cell number (division).

FIG. 8. Cytokinin Treatment. Data from an experiment showing that the ZmCNR02 genes' expression, as revealed by MPSS transcript assay, decreases in excised maize leaf discs, when 10 micromolar benzyladenine is added for 6 hours. This result offers additional evidence that the expression of ZmCNR02 is consistent with a role in negatively regulating cell number. The addition of a plant hormone that is known to induce cell number (cell division) results in the decline in expression of ZmCNR02, as expected per the hypothesis that this gene negatively regulates cell number.

FIG. 9. ZmCNR02 Expression Negative Correlation with Growth: RT-PCR analysis of leaf sections of different growth activity in four genotypes. Leaf sections of different growth activity are collected from seedlings at V3 stage. The RT-PCR analysis of ZmCNR02 is multiplexed with tubulin as a control. RT-PCR analysis confirmed the negative correlation of ZmCNR02 expression with growth and activity in different expression platform from MPSS profiling. The negatively correlative relationship with growth is consistently seen in all four different genotypes tested, indicating the general role in growth of this gene regardless of the genetic backgrounds.

FIG. 10. ZmCNR02 Expression in Mature Leaf of inbred parents and their reciprocal hybrids. This is a RT-PCR assay with the mature leaf tissue, where the PCR protocol was modified to increase the amplification of tubulin that was out-competed by ZmCNR02's high expression. This figure shows well that the expression level of ZmCNR02 in both hybrids is significantly higher than the inbred parents.

### DETAILED DESCRIPTION OF THE INVENTION

Unless defined otherwise, all technical and scientific terms used herein have the same meaning as commonly understood by one of ordinary skill in the art to which this invention belongs. Unless mentioned otherwise, the techniques employed or contemplated herein are standard methodologies well known to one of ordinary skill in the art. The materials, methods and examples are illustrative only and not limiting. The following is presented by way of illustration and is not intended to limit the scope of the invention.

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature. See, e.g., Langenheim and Thimann, BOTANY: PLANT BIOLOGY AND ITS RELATION TO HUMAN AFFAIRS, John Wiley (1982); CELL CULTURE AND SOMATIC CELL GENETICS OF PLANTS, vol. 1, Vasil, ed. (1984); Stanier, et al., THE MICROBIAL WORLD, 5^{th} ed., Prentice-Hall (1986); Dhringra and Sinclair, BASIC PLANT PATHOLOGY METHODS, CRC Press (1985); Maniatis, et al., MOLECULAR CLONING: A LABORATORY MANUAL (1982); DNA CLONING, vols. I and II, Glover, ed. (1985); OLIGONUCLEOTIDE SYNTHESIS, Gait, ed. (1984); NUCLEIC ACID HYBRIDIZATION, Hames and Higgins, eds. (1984); and the series METHODS IN ENZYMOLOGY, Colowick and Kaplan, eds, Academic Press, Inc., San Diego, Calif.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The terms defined below are more fully defined by reference to the specification as a whole.

In describing the present invention, the following terms will be employed, and are intended to be defined as indicated below.

By “microbe” is meant any microorganism (including both eukaryotic and prokaryotic microorganisms), such as fungi, yeast, bacteria, actinomycetes, algae and protozoa, as well as other unicellular structures.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS) and strand displacement amplification (SDA). See, e.g., DIAGNOSTIC MOLECULAR MICROBIOLOGY: PRINCIPLES AND APPLICATIONS, Persing et al., eds., American Society for Microbiology, Washington, D.C. (1993). The product of amplification is termed an amplicon.

The term “conservatively modified variants” applies to both amino acid and nucleic acid sequences. With respect to particular nucleic acid sequences, conservatively modified variants refer to those nucleic acids that encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also describes every possible silent variation of the nucleic acid. One of ordinary skill will recognize that each codon in a nucleic acid (except AUG, which is ordinarily the only codon for methionine; one exception is Micrococcus rubens, for which GTG is the methionine codon (Ishizuka, et al., (1993) J. Gen. Microbiol. 139:425-32) can be modified to yield a functionally identical molecule. Accordingly, each silent variation of a nucleic acid, which encodes a polypeptide of the present invention, is implicit in each described polypeptide sequence and incorporated herein by reference.

As to amino acid sequences, one of skill will recognize that individual substitutions, deletions or additions to a nucleic acid, peptide, polypeptide or protein sequence which alters, adds or deletes a single amino acid or a small percentage of amino acids in the encoded sequence is a “conservatively modified variant” when the alteration results in the substitution of an amino acid with a chemically similar amino acid. Thus, any number of amino acid residues selected from the group of integers consisting of from 1 to 15 can be so altered. Thus, for example, 1, 2, 3, 4, 5, 7 or 10 alterations can be made. Conservatively modified variants typically provide similar biological activity as the unmodified polypeptide sequence from which they are derived. For example, substrate specificity, enzyme activity, or ligand/receptor binding is generally at least 30%, 40%, 50%, 60%, 70%, 80% or 90%, preferably 60-90% of the native protein for it's native substrate. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

See also, Creighton, PROTEINS, W.H. Freeman and Co. (1984).

As used herein, “consisting essentially of” means the inclusion of additional sequences to an object polynucleotide where the additional sequences do not selectively hybridize, under stringent hybridization conditions, to the same cDNA as the polynucleotide and where the hybridization conditions include a wash step in 0.1×SSC and 0.1% sodium dodecyl sulfate at 65° C.

By “encoding” or “encoded,” with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as is present in some plant, animal, and fungal mitochondria, the bacterium Mycoplasma capricolumn (Yamao, et al., (1985) Proc. Natl. Acad. Sci. USA 82:2306-9) or the ciliate Macronucleus, may be used when the nucleic acid is expressed using these organisms.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledonous plants or dicotyledonous plants as these preferences have been shown to differ (Murray, et al., (1989) Nucleic Acids Res. 17:477-98 and herein incorporated by reference). Thus, the maize preferred codon for a particular amino acid might be derived from known gene sequences from maize. Maize codon usage for 28 genes from maize plants is listed in Table 4 of Murray et al., supra.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived or, if from the same species, one or both are substantially modified from their original form. A heterologous protein may originate from a foreign species or, if from the same species, is substantially modified from its original form by deliberate human intervention.

By “host cell” is meant a cell, which comprises a heterologous nucleic acid sequence of the invention, which contains a vector and supports the replication and/or expression of the expression vector. Host cells may be prokaryotic cells such as E. coli, or eukaryotic cells such as yeast, insect, plant, amphibian or mammalian cells. Preferably, host cells are monocotyledonous or dicotyledonous plant cells, including but not limited to maize, sorghum, sunflower, soybean, wheat, alfalfa, rice, cotton, canola, barley, millet and tomato. A particularly preferred monocotyledonous host cell is a maize host cell.

The term “hybridization complex” includes reference to a duplex nucleic acid structure formed by two single-stranded nucleic acid sequences selectively hybridized with each other.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The terms “isolated” refers to material, such as a nucleic acid or a protein, which is substantially or essentially free from components which normally accompany or interact with it as found in its naturally occurring environment. The isolated material optionally comprises material not found with the material in its natural environment. Nucleic acids, which are “isolated”, as defined herein, are also referred to as “heterologous” nucleic acids. Unless otherwise stated, the term “CNR nucleic acid” means a nucleic acid comprising a polynucleotide (“CNR polynucleotide”) encoding a full length or partial length CNR polypeptide.

As used herein, “nucleic acid” includes reference to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules, which comprise and substantially represent the entire transcribed fraction of a genome of a specified organism. Construction of exemplary nucleic acid libraries, such as genomic and cDNA libraries, is taught in standard molecular biology references such as Berger and Kimmel, GUIDE TO MOLECULAR CLONING TECHNIQUES, from the series METHODS IN ENZYMOLOGY, vol. 152, Academic Press, Inc., San Diego, Calif. (1987); Sambrook, et al., MOLECULAR CLONING: A LABORATORY MANUAL, 2^{nd} ed., vols. 1-3 (1989); and CURRENT PROTOCOLS IN MOLECULAR BIOLOGY, Ausubel, et al., eds, Current Protocols, a joint venture between Greene Publishing Associates, Inc. and John Wiley & Sons, Inc. (1994 Supplement).

As used herein “operably linked” includes reference to a functional linkage between a first sequence, such as a promoter, and a second sequence, wherein the promoter sequence initiates and mediates transcription of the DNA corresponding to the second sequence. Generally, operably linked means that the nucleic acid sequences being linked are contiguous and, where necessary to join two protein coding regions, contiguous and in the same reading frame.

As used herein, the term “plant” includes reference to whole plants, plant organs (e.g., leaves, stems, roots, etc.), seeds and plant cells and progeny of same. Plant cell, as used herein includes, without limitation, seeds, suspension cultures, embryos, meristematic regions, callus tissue, leaves, roots, shoots, gametophytes, sporophytes, pollen and microspores. The class of plants, which can be used in the methods of the invention, is generally as broad as the class of higher plants amenable to transformation techniques, including both monocotyledonous and dicotyledonous plants including species from the genera: Cucurbita, Rosa, Vitis, Juglans, Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Datura, Hyoscyamus, Lycopersicon, Nicotiana, Solanum, Petunia, Digitalis, Majorana, Ciahorium, Helianthus, Lactuca, Bromus, Asparagus, Antirrhinum, Heterocallis, Nemesis, Pelargonium, Panieum, Pennisetum, Ranunculus, Senecio, Salpiglossis, Cucumis, Browaalia, Glycine, Pisum, Phaseolus, Lolium, Oryza, Avena, Hordeum, Secale, Allium and Triticum. A particularly preferred plant is Zea mays.

As used herein, “yield” may include reference to bushels per acre of a grain crop at harvest, as adjusted for grain moisture (15% typically for maize, for example). Grain moisture is measured in the grain at harvest. The adjusted test weight of grain is determined to be the weight in pounds per bushel, adjusted for grain moisture level at harvest.

As used herein, “polynucleotide” includes reference to a deoxyribopolynucleotide, ribopolynucleotide or analogs thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells. Exemplary plant promoters include, but are not limited to, those that are obtained from plants, plant viruses, and bacteria which comprise genes expressed in plant cells such Agrobacterium or Rhizobium. Examples are promoters that preferentially initiate transcription in certain tissues, such as leaves, roots, seeds, fibres, xylem vessels, tracheids or sclerenchyma. Such promoters are referred to as “tissue preferred.” A “cell type” specific promoter primarily drives expression in certain cell types in one or more organs, for example, vascular cells in roots or leaves. An “inducible” or “regulatable” promoter is a promoter, which is under environmental control. Examples of environmental conditions that may effect transcription by inducible promoters include anaerobic conditions or the presence of light. Another type of promoter is a developmentally regulated promoter, for example, a promoter that drives expression during pollen development. Tissue preferred, cell type specific, developmentally regulated, and inducible promoters constitute the class of “non-constitutive” promoters. A “constitutive” promoter is a promoter, which is active under most environmental conditions.

The term “CNR polypeptide” refers to one or more amino acid sequences. The term is also inclusive of fragments, variants, homologs, alleles or precursors (e.g., preproproteins or proproteins) thereof. A “CNR protein” comprises a CNR polypeptide. Unless otherwise stated, the term “CNR nucleic acid” means a nucleic acid comprising a polynucleotide (“CNR polynucleotide”) encoding a CNR polypeptide.

As used herein “recombinant” includes reference to a cell or vector, that has been modified by the introduction of a heterologous nucleic acid or that the cell is derived from a cell so modified. Thus, for example, recombinant cells express genes that are not found in identical form within the native (non-recombinant) form of the cell or express native genes that are otherwise abnormally expressed, under expressed or not expressed at all as a result of deliberate human intervention; or may have reduced or eliminated expression of a native gene. The term “recombinant” as used herein does not encompass the alteration of the cell or vector by naturally occurring events (e.g., spontaneous mutation, natural transformation/transduction/transposition) such as those occurring without deliberate human intervention.

As used herein, a “recombinant expression cassette” is a nucleic acid construct, generated recombinantly or synthetically, with a series of specified nucleic acid elements, which permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “residue” or “amino acid residue” or “amino acid” are used interchangeably herein to refer to an amino acid that is incorporated into a protein, polypeptide or peptide (collectively “protein”). The amino acid may be a naturally occurring amino acid and, unless otherwise limited, may encompass known analogs of natural amino acids that can function in a similar manner as naturally occurring amino acids.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 40% sequence identity, preferably 60-90% sequence identity and most preferably 100% sequence identity (i.e., complementary) with each other.

The terms “stringent conditions” or “stringent hybridization conditions” include reference to conditions under which a probe will hybridize to its target sequence, to a detectably greater degree than other sequences (e.g., at least 2-fold over background). Stringent conditions are sequence-dependent and will be different in different circumstances. By controlling the stringency of the hybridization and/or washing conditions, target sequences can be identified which can be up to 100% complementary to the probe (homologous probing). Alternatively, stringency conditions can be adjusted to allow some mismatching in sequences so that lower degrees of similarity are detected (heterologous probing). Optimally, the probe is approximately 500 nucleotides in length, but can vary greatly in length from less than 500 nucleotides to equal to the entire length of the target sequence.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide or Denhardt's. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267-84 (1984): Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, LABORATORY TECHNIQUES IN BIOCHEMISTRY AND MOLECULAR BIOLOGY—HYBRIDIZATION WITH NUCLEIC ACID PROBES, part I, chapter 2, “Overview of principles of hybridization and the strategy of nucleic acid probe assays,” Elsevier, New York (1993); and CURRENT PROTOCOLS IN MOLECULAR BIOLOGY, chapter 2, Ausubel, et al., eds, Greene Publishing and Wiley-Interscience, New York (1995). Unless otherwise stated, in the present application high stringency is defined as hybridization in 4×SSC, 5×Denhardt's (5 g Ficoll, 5 g polyvinypyrrolidone, 5 g bovine serum albumin in 500 ml of water), 0.1 mg/ml boiled salmon sperm DNA, and 25 mM Na phosphate at 65° C., and a wash in 0.1×SSC, 0.1% SDS at 65° C.

As used herein, “transgenic plant” includes reference to a plant, which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition or spontaneous mutation.

As used herein, “vector” includes reference to a nucleic acid used in transfection of a host cell and into which can be inserted a polynucleotide. Vectors are often replicons. Expression vectors permit transcription of a nucleic acid inserted therein.

The following terms are used to describe the sequence relationships between two or more nucleic acids or polynucleotides or polypeptides: (a) “reference sequence,” (b) “comparison window,” (c) “sequence identity,” (d) “percentage of sequence identity” and (e) “substantial identity.”

As used herein, “reference sequence” is a defined sequence used as a basis for sequence comparison. A reference sequence may be a subset or the entirety of a specified sequence; for example, as a segment of a full-length cDNA or gene sequence or the complete cDNA or gene sequence.

As used herein, “comparison window” means includes reference to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100 or longer. Those of skill in the art understand that to avoid a high similarity to a reference sequence due to inclusion of gaps in the polynucleotide sequence a gap penalty is typically introduced and is subtracted from the number of matches.

Methods of alignment of nucleotide and amino acid sequences for comparison are well known in the art. The local homology algorithm (BESTFIT) of Smith and Waterman, Adv. Appl. Math 2:482 (1981), may conduct optimal alignment of sequences for comparison; by the homology alignment algorithm (GAP) of Needleman and Wunsch, J. Mol. Biol. 48:443-53 (1970); by the search for similarity method (Tfasta and Fasta) of Pearson and Lipman, Proc. Natl. Acad. Sci. USA 85:2444 (1988); by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG® programs (Accelrys, Inc., San Diego, Calif.)). The CLUSTAL program is well described by Higgins and Sharp, (1988) Gene 73:237-44; Higgins and Sharp, (1989) CABIOS 5:151-3; Corpet, et al., (1988) Nucleic Acids Res. 16:10881-90; Huang, et al., (1992) Computer Applications in the Biosciences 8:155-65 and Pearson, et al., (1994) Meth. Mol. Biol. 24:307-31. The preferred program to use for optimal global alignment of multiple sequences is PileUp (Feng and Doolittle, (1987) J. Mol. Evol., 25:351-60 which is similar to the method described by Higgins and Sharp, (1989) CABIOS 5:151-53 and hereby incorporated by reference). The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, CURRENT PROTOCOLS IN MOLECULAR BIOLOGY, Chapter 19, Ausubel, et al., eds., Greene Publishing and Wiley-Interscience, New York (1995).

GAP uses the algorithm of Needleman and Wunsch, supra, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or greater.

GAP presents one member of the family of best alignments. There may be many members of this family, but no other member has a better quality. GAP displays four figures of merit for alignments: Quality, Ratio, Identity and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see, Henikoff and Henikoff, (1989) Proc. Natl. Acad. Sci. USA 89:10915).

Unless otherwise stated, sequence identity/similarity values provided herein refer to the value obtained using the BLAST 2.0 suite of programs using default parameters (Altschul, et al., (1997) Nucleic Acids Res. 25:3389-402).

As those of ordinary skill in the art will understand, BLAST searches assume that proteins can be modeled as random sequences. However, many real proteins comprise regions of nonrandom sequences, which may be homopolymeric tracts, short-period repeats or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17:149-63) and XNU (Claverie and States, (1993) Comput. Chem. 17:191-201) low-complexity filters can be employed alone or in combination.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

Another indication that nucleotide sequences are substantially identical is if two molecules hybridize to each other under stringent conditions. The degeneracy of the genetic code allows for many amino acids substitutions that lead to variety in the nucleotide sequence that code for the same amino acid, hence it is possible that the DNA sequence could code for the same polypeptide but not hybridize to each other under stringent conditions. This may occur, e.g., when a copy of a nucleic acid is created using the maximum codon degeneracy permitted by the genetic code. One indication that two nucleic acid sequences are substantially identical is that the polypeptide, which the first nucleic acid encodes, is immunologically cross reactive with the polypeptide encoded by the second nucleic acid.

The terms “substantial identity” in the context of a peptide indicates that a peptide comprises a sequence with between 55-100% sequence identity to a reference sequence preferably at least 55% sequence identity, preferably 60% preferably 70%, more preferably 80%, most preferably at least 90% or 95% sequence identity to the reference sequence over a specified comparison window. Preferably, optimal alignment is conducted using the homology alignment algorithm of Needleman and Wunsch, supra. An indication that two peptide sequences are substantially identical is that one peptide is immunologically reactive with antibodies raised against the second peptide. Thus, a peptide is substantially identical to a second peptide, for example, where the two peptides differ only by a conservative substitution. In addition, a peptide can be substantially identical to a second peptide when they differ by a non-conservative change if the epitope that the antibody recognizes is substantially identical. Peptides, which are “substantially similar” share sequences as, noted above except that residue positions, which are not identical, may differ by conservative amino acid changes.

The invention discloses CNR polynucleotides and polypeptides. The novel nucleotides and proteins of the invention have an expression pattern which indicates that they regulate cell number and thus play an important role in plant development. The polynucleotides are expressed in various plant tissues. The polynucleotides and polypeptides thus provide an opportunity to manipulate plant development to alter seed and vegetative tissue development, timing or composition. This may be used to create a sterile plant, a seedless plant or a plant with altered endosperm composition.

### Nucleic Acids

The present invention provides, inter alia, isolated nucleic acids of RNA, DNA and analogs and/or chimeras thereof, comprising a CNR polynucleotide.

The present invention also includes polynucleotides optimized for expression in different organisms. For example, for expression of the polynucleotide in a maize plant, the sequence can be altered to account for specific codon preferences and to alter GC content as according to Murray, et al, supra. Maize codon usage for 28 genes from maize plants is listed in Table 4 of Murray, et al., supra.

The CNR nucleic acids of the present invention comprise isolated CNR polynucleotides which are inclusive of:
(a) a polynucleotide encoding a CNR polypeptide and conservatively modified and polymorphic variants thereof;
(b) a polynucleotide having at least 70% sequence identity with polynucleotides of (a) or (b);
(c) complementary sequences of polynucleotides of (a) or (b).

### Construction of Nucleic Acids

The isolated nucleic acids of the present invention can be made using (a) standard recombinant methods, (b) synthetic techniques, or combinations thereof. In some embodiments, the polynucleotides of the present invention will be cloned, amplified or otherwise constructed from a fungus or bacteria.

The nucleic acids may conveniently comprise sequences in addition to a polynucleotide of the present invention. For example, a multi-cloning site comprising one or more endonuclease restriction sites may be inserted into the nucleic acid to aid in isolation of the polynucleotide. Also, translatable sequences may be inserted to aid in the isolation of the translated polynucleotide of the present invention. For example, a hexa-histidine marker sequence provides a convenient means to purify the proteins of the present invention. The nucleic acid of the present invention—excluding the polynucleotide sequence—is optionally a vector, adapter, or linker for cloning and/or expression of a polynucleotide of the present invention. Additional sequences may be added to such cloning and/or expression sequences to optimize their function in cloning and/or expression, to aid in isolation of the polynucleotide, or to improve the introduction of the polynucleotide into a cell. Typically, the length of a nucleic acid of the present invention less the length of its polynucleotide of the present invention is less than 20 kilobase pairs, often less than 15 kb and frequently less than 10 kb. Use of cloning vectors, expression vectors, adapters, and linkers is well known in the art. Exemplary nucleic acids include such vectors as: M13, lambda ZAP Express, lambda ZAP II, lambda gt10, lambda gt11, pBK-CMV, pBK-RSV, pBluescript II, lambda DASH II, lambda EMBL 3, lambda EMBL 4, pWE15, SuperCos 1, SurfZap, Uni-ZAP, pBC, pBS+/−, pSG5, pBK, pCR-Script, pET, pSPUTK, p3'SS, pGEM, pSK+/−, pGEX, pSPORTI and II, pOPRSVI CAT, pOPI3 CAT, pXT1, pSG5, pPbac, pMbac, pMC1neo, pOG44, pOG45, pFRTβGAL, pNEOβGAL, pRS403, pRS404, pRS405, pRS406, pRS413, pRS414, pRS415, pRS416, lambda MOSSIox and lambda MOSEIox. Optional vectors for the present invention, include but are not limited to, lambda ZAP II and pGEX. For a description of various nucleic acids. see, e.g., Stratagene Cloning Systems, Catalogs 1995, 1996, 1997 (La Jolla, Calif.); and, Amersham Life Sciences, Inc, Catalog '97 (Arlington Heights, Ill.).

### Synthetic Methods for Constructing Nucleic Acids

The isolated nucleic acids of the present invention can also be prepared by direct chemical synthesis by methods such as the phosphotriester method of Narang, et al., (1979) Meth. Enzymol. 68:90-9; the phosphodiester method of Brown, et al., (1979) Meth. Enzymol. 68:109-51; the diethylphosphoramidite method of Beaucage, et al., (1981) Tetra. Letts. 22(20):1859-62; the solid phase phosphoramidite triester method described by Beaucage, et al., supra, e.g., using an automated synthesizer, e.g., as described in Needham-VanDevanter, et al., (1984) Nucleic Acids Res. 12:6159-68; and, the solid support method of U.S. Pat. No. 4,458,066. Chemical synthesis generally produces a single stranded oligonucleotide. This may be converted into double stranded DNA by hybridization with a complementary sequence or by polymerization with a DNA polymerase using the single strand as a template. One of skill will recognize that while chemical synthesis of DNA is limited to sequences of about 100 bases, longer sequences may be obtained by the ligation of shorter sequences.

### UTRs and Codon Preference

In general, translational efficiency has been found to be regulated by specific sequence elements in the 5′ non-coding or untranslated region (5′ UTR) of the RNA. Positive sequence motifs include translational initiation consensus sequences (Kozak, (1987) Nucleic Acids Res. 15:8125) and the 5<G>7 methyl GpppG RNA cap structure (Drummond, et al., (1985) Nucleic Acids Res. 13:7375). Negative elements include stable intramolecular 5′ UTR stem-loop structures (Muesing, et al., (1987) Cell 48:691) and AUG sequences or short open reading frames preceded by an appropriate AUG in the 5′ UTR (Kozak, supra, Rao, et al., (1988) Mol. and Cell. Biol. 8:284). Accordingly, the present invention provides 5′ and/or 3′ UTR regions for modulation of translation of heterologous coding sequences.

Further, the polypeptide-encoding segments of the polynucleotides of the present invention can be modified to alter codon usage. Altered codon usage can be employed to alter translational efficiency and/or to optimize the coding sequence for expression in a desired host or to optimize the codon usage in a heterologous sequence for expression in maize. Codon usage in the coding regions of the polynucleotides of the present invention can be analyzed statistically using commercially available software packages such as “Codon Preference” available from the University of Wisconsin Genetics Computer Group. See, Devereaux, et al., (1984) Nucleic Acids Res. 12:387-395) or MacVector 4.1 (Eastman Kodak Co., New Haven, Conn.). Thus, the present invention provides a codon usage frequency characteristic of the coding region of at least one of the polynucleotides of the present invention. The number of polynucleotides (3 nucleotides per amino acid) that can be used to determine a codon usage frequency can be any integer from 3 to the number of polynucleotides of the present invention as provided herein. Optionally, the polynucleotides will be full-length sequences. An exemplary number of sequences for statistical analysis can be at least 1, 5, 10, 20, 50 or 100.

### Sequence Shuffling

The present invention provides methods for sequence shuffling using polynucleotides of the present invention, and compositions resulting therefrom. Sequence shuffling is described in PCT Publication Number 96/19256. See also, Zhang, et al., (1997) Proc. Natl. Acad. Sci. USA 94:4504-9 and Zhao, et al., (1998) Nature Biotech 16:258-61. Generally, sequence shuffling provides a means for generating libraries of polynucleotides having a desired characteristic, which can be selected or screened for. Libraries of recombinant polynucleotides are generated from a population of related sequence polynucleotides, which comprise sequence regions, which have substantial sequence identity and can be homologously recombined in vitro or in vivo. The population of sequence-recombined polynucleotides comprises a subpopulation of polynucleotides which possess desired or advantageous characteristics and which can be selected by a suitable selection or screening method. The characteristics can be any property or attribute capable of being selected for or detected in a screening system, and may include properties of: an encoded protein, a transcriptional element, a sequence controlling transcription, RNA processing, RNA stability, chromatin conformation, translation or other expression property of a gene or transgene, a replicative element, a protein-binding element or the like, such as any feature which confers a selectable or detectable property. In some embodiments, the selected characteristic will be an altered Kₘ and/or K_{cat} over the wild-type protein as provided herein. In other embodiments, a protein or polynucleotide generated from sequence shuffling will have a ligand binding affinity greater than the non-shuffled wild-type polynucleotide. In yet other embodiments, a protein or polynucleotide generated from sequence shuffling will have an altered pH optimum as compared to the non-shuffled wild-type polynucleotide. The increase in such properties can be at least 110%, 120%, 130%, 140% or greater than 150% of the wild-type value.

### Recombinant Expression Cassettes

The present invention further provides recombinant expression cassettes comprising a nucleic acid of the present invention. A nucleic acid sequence coding for the desired polynucleotide of the present invention, for example a cDNA or a genomic sequence encoding a polypeptide long enough to code for an active protein of the present invention, can be used to construct a recombinant expression cassette which can be introduced into the desired host cell. A recombinant expression cassette will typically comprise a polynucleotide of the present invention operably linked to transcriptional initiation regulatory sequences which will direct the transcription of the polynucleotide in the intended host cell, such as tissues of a transformed plant.

For example, plant expression vectors may include (1) a cloned plant gene under the transcriptional control of 5′ and 3′ regulatory sequences and (2) a dominant selectable marker. Such plant expression vectors may also contain, if desired, a promoter regulatory region (e.g., one conferring inducible or constitutive, environmentally- or developmentally-regulated, or cell- or tissue-specific/selective expression), a transcription initiation start site, a ribosome binding site, an RNA processing signal, a transcription termination site and/or a polyadenylation signal.

A plant promoter fragment can be employed which will direct expression of a polynucleotide of the present invention in all tissues of a regenerated plant. Such promoters are referred to herein as “constitutive” promoters and are active under most environmental conditions and states of development or cell differentiation. Examples of constitutive promoters include the 1′- or 2′-promoter derived from T-DNA of Agrobacterium tumefaciens, the Smas promoter, the cinnamyl alcohol dehydrogenase promoter (U.S. Pat. No. 5,683,439), the Nos promoter, the rubisco promoter, the GRP1-8 promoter, the ³⁵S promoter from cauliflower mosaic virus (CaMV), as described in Odell, et al., (1985) Nature 313:810-2; rice actin (McElroy, et al., (1990) Plant Cell 163-171); ubiquitin (Christensen, et al., (1992) Plant Mol. Biol. 12:619-632 and Christensen, et al., (1992) Plant Mol. Biol. 18:675-89); pEMU (Last, et al., (1991) Theor. Appl. Genet. 81:581-8); MAS (Velten, et al., (1984) EMBO J. 3:2723-30); and maize H3 histone (Lepetit, et al., (1992) Mol. Gen. Genet. 231:276-85 and Atanassvoa, et al., (1992) Plant Journal 2(3):291-300); ALS promoter, as described in PCT Application Number WO 96/30530; and other transcription initiation regions from various plant genes known to those of skill. For the present invention ubiquitin is the preferred promoter for expression in monocot plants.

Alternatively, the plant promoter can direct expression of a polynucleotide of the present invention in a specific tissue or may be otherwise under more precise environmental or developmental control. Such promoters are referred to here as “inducible” promoters. Environmental conditions that may effect transcription by inducible promoters include pathogen attack, anaerobic conditions, or the presence of light. Examples of inducible promoters are the Adh1 promoter, which is inducible by hypoxia or cold stress, the Hsp70 promoter, which is inducible by heat stress, and the PPDK promoter, which is inducible by light.

Examples of promoters under developmental control include promoters that initiate transcription only, or preferentially, in certain tissues, such as leaves, roots, fruit, seeds or flowers. The operation of a promoter may also vary depending on its location in the genome. Thus, an inducible promoter may become fully or partially constitutive in certain locations.

If polypeptide expression is desired, it is generally desirable to include a polyadenylation region at the 3′-end of a polynucleotide coding region. The polyadenylation region can be derived from a variety of plant genes, or from T-DNA. The 3′ end sequence to be added can be derived from, for example, the nopaline synthase or octopine synthase genes, or alternatively from another plant gene, or less preferably from any other eukaryotic gene. Examples of such regulatory elements include, but are not limited to, 3′ termination and/or polyadenylation regions such as those of the Agrobacterium tumefaciens nopaline synthase (nos) gene (Bevan, et al., (1983) Nucleic Acids Res. 12:369-85); the potato proteinase inhibitor II (PINII) gene (Keil, et al., (1986) Nucleic Acids Res. 14:5641-50; and An, et al., (1989) Plant Cell 1:115-22); and the CaMV 19S gene (Mogen, et al., 1990) Plant Cell 2:1261-72).

An intron sequence can be added to the 5′ untranslated region or the coding sequence of the partial coding sequence to increase the amount of the mature message that accumulates in the cytosol. Inclusion of a spliceable intron in the transcription unit in both plant and animal expression constructs has been shown to increase gene expression at both the mRNA and protein levels up to 1000-fold (Buchman and Berg, (1988) Mol. Cell Biol. 8:4395-4405; Callis, et al., (1987) Genes Dev. 1:1183-200). Such intron enhancement of gene expression is typically greatest when placed near the 5′ end of the transcription unit. Use of maize introns Adh1-S intron 1, 2 and 6, the Bronze-1 intron are known in the art. See generally, THE MAIZE HANDBOOK, Chapter 116, Freeling and Walbot, eds., Springer, New York (1994).

Plant signal sequences, including, but not limited to, signal-peptide encoding DNA/RNA sequences which target proteins to the extracellular matrix of the plant cell (Dratewka-Kos, et al., (1989) J. Biol. Chem. 264:4896-900), such as the Nicotiana plumbaginifolia extension gene (DeLoose, et al., (1991) Gene 99:95-100); signal peptides which target proteins to the vacuole, such as the sweet potato sporamin gene (Matsuka, et al., (1991) Proc. Natl. Acad. Sci. USA 88:834) and the barley lectin gene (Wilkins, et al., (1990) Plant Cell, 2:301-13); signal peptides which cause proteins to be secreted, such as that of PRIb (Lind, et al., (1992) Plant Mol. Biol. 18:47-53) or the barley alpha amylase (BAA) (Rahmatullah, et al., (1989) Plant Mol. Biol. 12:119, and hereby incorporated by reference), or signal peptides which target proteins to the plastids such as that of rapeseed enoyl-Acp reductase (Verwaert, et al., (1994) Plant Mol. Biol. 26:189-202) are useful in the invention. The barley alpha amylase signal sequence fused to the CNR polynucleotide is the preferred construct for expression in maize for the present invention.

The vector comprising the sequences from a polynucleotide of the present invention will typically comprise a marker gene, which confers a selectable phenotype on plant cells. Usually, the selectable marker gene will encode antibiotic resistance, with suitable genes including genes coding for resistance to the antibiotic spectinomycin (e.g., the aada gene), the streptomycin phosphotransferase (SPT) gene coding for streptomycin resistance, the neomycin phosphotransferase (NPTII) gene encoding kanamycin or geneticin resistance, the hygromycin phosphotransferase (HPT) gene coding for hygromycin resistance, genes coding for resistance to herbicides which act to inhibit the action of acetolactate synthase (ALS), in particular the sulfonylurea-type herbicides (e.g., the acetolactate synthase (ALS) gene containing mutations leading to such resistance in particular the S4 and/or Hra mutations), genes coding for resistance to herbicides which act to inhibit action of glutamine synthase, such as phosphinothricin or basta (e.g., the bar gene) or other such genes known in the art. The bar gene encodes resistance to the herbicide basta, and the ALS gene encodes resistance to the herbicide chlorsulfuron.

Typical vectors useful for expression of genes in higher plants are well known in the art and include vectors derived from the tumor-inducing (Ti) plasmid of Agrobacterium tumefaciens described by Rogers, et al., (1987) Meth. Enzymol. 153:253-77. These vectors are plant integrating vectors in that on transformation, the vectors integrate a portion of vector DNA into the genome of the host plant. Exemplary A. tumefaciens vectors useful herein are plasmids pKYLX6 and pKYLX7 of Schardl, et al., (1987) Gene 61:1-11, and Berger, et al., (1989) Proc. Natl. Acad. Sci. USA, 86:8402-6. Another useful vector herein is plasmid pBI101.2 that is available from CLONTECH Laboratories, Inc. (Palo Alto, Calif.).

### Expression of Proteins in Host Cells

Using the nucleic acids of the present invention, one may express a protein of the present invention in a recombinantly engineered cell such as bacteria, yeast, insect, mammalian or preferably plant cells. The cells produce the protein in a non-natural condition (e.g., in quantity, composition, location and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible), followed by incorporation into an expression vector. The vectors can be suitable for replication and integration in either prokaryotes or eukaryotes. Typical expression vectors contain transcription and translation terminators, initiation sequences, and promoters useful for regulation of the expression of the DNA encoding a protein of the present invention. To obtain high level expression of a cloned gene, it is desirable to construct expression vectors which contain, at the minimum, a strong promoter, such as ubiquitin, to direct transcription, a ribosome binding site for translational initiation and a transcription/translation terminator. Constitutive promoters are classified as providing for a range of constitutive expression. Thus, some are weak constitutive promoters and others are strong constitutive promoters. Generally, by “weak promoter” is intended a promoter that drives expression of a coding sequence at a low level. By “low level” is intended at levels of about 1/10,000 transcripts to about 1/100,000 transcripts to about 1/500,000 transcripts. Conversely, a “strong promoter” drives expression of a coding sequence at a “high level” or about 1/10 transcripts to about 1/100 transcripts to about 1/1,000 transcripts.

One of skill would recognize that modifications could be made to a protein of the present invention without diminishing its biological activity. Some modifications may be made to facilitate the cloning, expression or incorporation of the targeting molecule into a fusion protein. Such modifications are well known to those of skill in the art and include, for example, a methionine added at the amino terminus to provide an initiation site or additional amino acids (e.g., poly His) placed on either terminus to create conveniently located restriction sites or termination codons or purification sequences.

### Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., (1977) Nature 198:1056), the tryptophan (trp) promoter system (Goeddel, et al., (1980) Nucleic Acids Res. 8:4057) and the lambda derived P L promoter and N-gene ribosome binding site (Shimatake, et al., (1981) Nature 292:128). The inclusion of selection markers in DNA vectors transfected in E. coli is also useful. Examples of such markers include genes specifying resistance to ampicillin, tetracycline, or chloramphenicol.

The vector is selected to allow introduction of the gene of interest into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva, et al., (1983) Gene 22:229-35; Mosbach, et al., (1983) Nature 302:543-5). The pGEX-4T-1 plasmid vector from Pharmacia is the preferred E. coli expression vector for the present invention.

### Expression in Eukaryotes

A variety of eukaryotic expression systems such as yeast, insect cell lines, plant and mammalian cells, are known to those of skill in the art. As explained briefly below, the present invention can be expressed in these eukaryotic systems. In some embodiments, transformed/transfected plant cells, as discussed infra, are employed as expression systems for production of the proteins of the instant invention.

Synthesis of heterologous proteins in yeast is well known. Sherman, et al., METHODS IN YEAST GENETICS, Cold Spring Harbor Laboratory (1982) is a well recognized work describing the various methods available to produce the protein in yeast. Two widely utilized yeasts for production of eukaryotic proteins are Saccharomyces cerevisiae and Pichia pastoris. Vectors, strains and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase and an origin of replication, termination sequences and the like as desired.

A protein of the present invention, once expressed, can be isolated from yeast by lysing the cells and applying standard protein isolation techniques to the lysates or the pellets. The monitoring of the purification process can be accomplished by using Western blot techniques or radioimmunoassay of other standard immunoassay techniques.

The sequences encoding proteins of the present invention can also be ligated to various expression vectors for use in transfecting cell cultures of, for instance, mammalian, insect or plant origin. Mammalian cell systems often will be in the form of monolayers of cells although mammalian cell suspensions may also be used. A number of suitable host cell lines capable of expressing intact proteins have been developed in the art, and include the HEK293, BHK21, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., (1986) Immunol. Rev. 89:49) and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection Catalogue of Cell Lines and Hybridomas (7^{th} ed., 1992).

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (see, e.g., Schneider, (1987) J. Embryol. Exp. Morphol. 27:353-65).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al., (1983) J. Virol. 45:773-81). Additionally, gene sequences to control replication in the host cell may be incorporated into the vector such as those found in bovine papilloma virus type-vectors (Saveria-Campo, “Bovine Papilloma Virus DNA a Eukaryotic Cloning Vector,” in DNA CLONING: A PRACTICAL APPROACH, vol. II, Glover, ed., IRL Press, Arlington, Va., pp. 213-38 (1985)).

In addition, the gene for CNR placed in the appropriate plant expression vector can be used to transform plant cells. The polypeptide can then be isolated from plant callus or the transformed cells can be used to regenerate transgenic plants. Such transgenic plants can be harvested, and the appropriate tissues (seed or leaves, for example) can be subjected to large scale protein extraction and purification techniques.

### Plant Transformation Methods

Numerous methods for introducing foreign genes into plants are known and can be used to insert a CNR polynucleotide into a plant host, including biological and physical plant transformation protocols. See, e.g., Miki, et al., (1993) “Procedure for Introducing Foreign DNA into Plants,” in METHODS IN PLANT MOLECULAR BIOLOGY AND BIOTECHNOLOGY, Glick and Thompson, eds., CRC Press, Inc., Boca Raton, pp. 67-88. The methods chosen vary with the host plant, and include chemical transfection methods such as calcium phosphate, microorganism-mediated gene transfer such as Agrobacterium (Horsch, et al., (1985) Science 227:1229-31), electroporation, micro-injection and biolistic bombardment.

Expression cassettes and vectors and in vitro culture methods for plant cell or tissue transformation and regeneration of plants are known and available. See, e.g., Gruber, et al., “Vectors for Plant Transformation,” in METHODS IN PLANT MOLECULAR BIOLOGY AND BIOTECHNOLOGY, supra, pp. 89-119.

The isolated polynucleotides or polypeptides may be introduced into the plant by one or more techniques typically used for direct delivery into cells. Such protocols may vary depending on the type of organism, cell, plant or plant cell, i.e., monocot or dicot, targeted for gene modification. Suitable methods of transforming plant cells include microinjection (Crossway, et al., (1986) Biotechniques 4:320-334 and U.S. Pat. No. 6,300,543), electroporation (Riggs, et al., (1986) Proc. Natl. Acad. Sci. USA 83:5602-5606, direct gene transfer (Paszkowski, et al., (1984) EMBO J. 3:2717-2722) and ballistic particle acceleration (see, for example, Sanford, et al., U.S. Pat. No. 4,945,050; WO 1991/10725 and McCabe, et al., (1988) Biotechnology 6:923-926). Also see, Tomes, et al., Direct DNA Transfer into Intact Plant Cells Via Microprojectile Bombardment. pp. 197-213 in Plant Cell, Tissue and Organ Culture, Fundamental Methods eds. Gamborg and Phillips. Springer-Verlag Berlin Heidelberg New York, 1995; U.S. Pat. No. 5,736,369 (meristem); Weissinger, et al., (1988) Ann. Rev. Genet. 22:421-477; Sanford, et al., (1987) Particulate Science and Technology 5:27-37 (onion); Christou, et al., (1988) Plant Physiol. 87:671-674 (soybean); Datta, et al., (1990) Biotechnology 8:736-740 (rice); Klein, et al., (1988) Proc. Natl. Acad. Sci. USA 85:4305-4309 (maize); Klein, et al., (1988) Biotechnology 6:559-563 (maize); WO 91/10725 (maize); Klein, et al., (1988) Plant Physiol. 91:440-444 (maize); Fromm, et al., (1990) Biotechnology 8:833-839 and Gordon-Kamm, et al., (1990) Plant Cell 2:603-618 (maize); Hooydaas-Van Slogteren and Hooykaas, (1984) Nature (London) 311:763-764; Bytebier, et al., (1987) Proc. Natl. Acad. Sci. USA 84:5345-5349 (Liliaceae); De Wet, et al., (1985) In The Experimental Manipulation of Ovule Tissues, ed. Chapman, et al., pp. 197-209. Longman, N.Y. (pollen); Kaeppler, et al., (1990) Plant Cell Reports 9:415-418 and Kaeppler, et al., (1992) Theor. Appl. Genet. 84:560-566 (whisker-mediated transformation); U.S. Pat. No. 5,693,512 (sonication); D'Halluin, et al., (1992) Plant Cell 4:1495-1505 (electroporation); Li, et al., (1993) Plant Cell Reports 12:250-255 and Christou and Ford, (1995) Annals of Botany 75:407-413 (rice); Osjoda, et al., (1996) Nature Biotech. 14:745-750; Agrobacterium mediated maize transformation (U.S. Pat. No. 5,981,840); silicon carbide whisker methods (Frame, et al., (1994) Plant J. 6:941-948); laser methods (Guo, et al., (1995) Physiologia Plantarum 93:19-24); sonication methods (Bao, et al., (1997) Ultrasound in Medicine and Biology 23:953-959; Finer and Finer, (2000) Lett Appl Microbiol. 30:406-10; Amoah, et al., (2001) J Exp Bot 52:1135-42); polyethylene glycol methods (Krens, et al., (1982) Nature 296:72-77); protoplasts of monocot and dicot cells can be transformed using electroporation (Fromm, et al., (1985) Proc. Natl. Acad. Sci. USA 82:5824-5828) and microinjection (Crossway, et al., (1986) Mol. Gen. Genet. 202:179-185); all of which are herein incorporated by reference.

### Agrobacterium-Mediated Transformation

The most widely utilized method for introducing an expression vector into plants is based on the natural transformation system of Agrobacterium. A. tumefaciens and A. rhizogenes are plant pathogenic soil bacteria, which genetically transform plant cells. The Ti and Ri plasmids of A. tumefaciens and A. rhizogenes, respectively, carry genes responsible for genetic transformation of plants. See, e.g., Kado, (1991) Crit. Rev. Plant Sci. 10:1. Descriptions of the Agrobacterium vector systems and methods for Agrobacterium-mediated gene transfer are provided in Gruber, et al., supra; Miki, et al., supra and Moloney, et al., (1989) Plant Cell Reports 8:238.

Similarly, the gene can be inserted into the T-DNA region of a Ti or Ri plasmid derived from A. tumefaciens or A. rhizogenes, respectively. Thus, expression cassettes can be constructed as above, using these plasmids. Many control sequences are known which when coupled to a heterologous coding sequence and transformed into a host organism show fidelity in gene expression with respect to tissue/organ specificity of the original coding sequence. See, e.g., Benfey and Chua, (1989) Science 244:174-81. Particularly suitable control sequences for use in these plasmids are promoters for constitutive leaf-specific expression of the gene in the various target plants. Other useful control sequences include a promoter and terminator from the nopaline synthase gene (NOS). The NOS promoter and terminator are present in the plasmid pARC2, available from the American Type Culture Collection and designated ATCC 67238. If such a system is used, the virulence (vir) gene from either the Ti or Ri plasmid must also be present, either along with the T-DNA portion, or via a binary system where the vir gene is present on a separate vector. Such systems, vectors for use therein, and methods of transforming plant cells are described in U.S. Pat. No. 4,658,082; U.S. patent application Ser. No. 913,914, filed Oct. 1, 1986, as referenced in U.S. Pat. No. 5,262,306, issued Nov. 16, 1993 and Simpson, et al., (1986) Plant Mol. Biol. 6: 403-15 (also referenced in the '306 patent); all incorporated by reference in their entirety.

Once constructed, these plasmids can be placed into A. rhizogenes or A. tumefaciens and these vectors used to transform cells of plant species, which are ordinarily susceptible to Fusarium or Alternaria infection. Several other transgenic plants are also contemplated by the present invention including but not limited to soybean, corn, sorghum, alfalfa, rice, clover, cabbage, banana, coffee, celery, tobacco, cowpea, cotton, melon and pepper. The selection of either A. tumefaciens or A. rhizogenes will depend on the plant being transformed thereby. In general A. tumefaciens is the preferred organism for transformation. Most dicotyledonous plants, some gymnosperms, and a few monocotyledonous plants (e.g., certain members of the Liliales and Arales) are susceptible to infection with A. tumefaciens. A. rhizogenes also has a wide host range, embracing most dicots and some gymnosperms, which includes members of the Leguminosae, Compositae and Chenopodiaceae. Monocot plants can now be transformed with some success. EP Patent Application Number 604 662 A1 discloses a method for transforming monocots using Agrobacterium. EP Application Number 672 752 A1 discloses a method for transforming monocots with Agrobacterium using the scutellum of immature embryos. Ishida, et al., discuss a method for transforming maize by exposing immature embryos to A. tumefaciens (Nature Biotechnology 14:745-50 (1996)).

Once transformed, these cells can be used to regenerate transgenic plants. For example, whole plants can be infected with these vectors by wounding the plant and then introducing the vector into the wound site. Any part of the plant can be wounded, including leaves, stems and roots. Alternatively, plant tissue, in the form of an explant, such as cotyledonary tissue or leaf disks, can be inoculated with these vectors, and cultured under conditions, which promote plant regeneration. Roots or shoots transformed by inoculation of plant tissue with A. rhizogenes or A. tumefaciens, containing the gene coding for the fumonisin degradation enzyme, can be used as a source of plant tissue to regenerate fumonisin-resistant transgenic plants, either via somatic embryogenesis or organogenesis. Examples of such methods for regenerating plant tissue are disclosed in Shahin, Theor. Appl. Genet. 69:235-40 (1985); U.S. Pat. No. 4,658,082; Simpson, et al., supra and U.S. patent application Ser. Nos. 913,913 and 913,914, both filed Oct. 1, 1986, as referenced in U.S. Pat. No. 5,262,306, issued Nov. 16, 1993, the entire disclosures therein incorporated herein by reference.

### Direct Gene Transfer

Despite the fact that the host range for Agrobacterium-mediated transformation is broad, some major cereal crop species and gymnosperms have generally been recalcitrant to this mode of gene transfer, even though some success has recently been achieved in rice (Hiei, et al., (1994) The Plant Journal 6:271-82). Several methods of plant transformation, collectively referred to as direct gene transfer, have been developed as an alternative to Agrobacterium-mediated transformation.

A generally applicable method of plant transformation is microprojectile-mediated transformation, where DNA is carried on the surface of microprojectiles measuring about 1 to 4 μm. The expression vector is introduced into plant tissues with a biolistic device that accelerates the microprojectiles to speeds of 300 to 600 m/s which is sufficient to penetrate the plant cell walls and membranes (Sanford, et al., (1987) Part. Sci. Technol. 5:27; Sanford, (1988) Trends Biotech 6:299; Sanford, (1990) Physiol. Plant 79:206; and Klein, et al., (1992) Biotechnology 10:268).

Another method for physical delivery of DNA to plants is sonication of target cells as described in Zang, et al., (1991) BioTechnology 9:996. Alternatively, liposome or spheroplast fusions have been used to introduce expression vectors into plants. See, e.g., Deshayes, et al., (1985) EMBO J. 4:2731 and Christou, et al., (1987) Proc. Natl. Acad. Sci. USA 84:3962. Direct uptake of DNA into protoplasts using CaCl₂ precipitation, polyvinyl alcohol, or poly-L-ornithine has also been reported. See, e.g., Hain, et al., (1985) Mol. Gen. Genet. 199:161 and Draper, et al., (1982) Plant Cell Physiol. 23:451.

Electroporation of protoplasts and whole cells and tissues has also been described. See, e.g., Donn, et al., (1990) in Abstracts of the VIIth Int'l. Congress on Plant Cell and Tissue Culture IAPTC, A2-38, p. 53; D'Halluin, et al., (1992) Plant Cell 4:1495-505 and Spencer, et al., (1994) Plant Mol. Biol. 24:51-61.

Increasing the Activity and/or Level of a CNR Polypeptide

Methods are provided to increase the activity and/or level of the CNR polypeptide of the invention. An increase in the level and/or activity of the CNR polypeptide of the invention can be achieved by providing to the plant a CNR polypeptide. The CNR polypeptide can be provided by introducing the amino acid sequence encoding the CNR polypeptide into the plant, introducing into the plant a nucleotide sequence encoding a CNR polypeptide or alternatively by modifying a genomic locus encoding the CNR polypeptide of the invention.

As discussed elsewhere herein, many methods are known the art for providing a polypeptide to a plant including, but not limited to, direct introduction of the polypeptide into the plant, introducing into the plant (transiently or stably) a polynucleotide construct encoding a polypeptide having cell number regulator activity. It is also recognized that the methods of the invention may employ a polynucleotide that is not capable of directing, in the transformed plant, the expression of a protein or an RNA. Thus, the level and/or activity of a CNR polypeptide may be increased by altering the gene encoding the CNR polypeptide or its promoter. See, e.g., Kmiec, U.S. Pat. No. 5,565,350; Zarling, et al., PCT/US93/03868. Therefore mutagenized plants that carry mutations in CNR genes, where the mutations increase expression of the CNR gene or increase the cell number regulator activity of the encoded CNR polypeptide are provided.

Reducing the Activity and/or Level of a CNR Polypeptide

Methods are provided to reduce or eliminate the activity of a CNR polypeptide of the invention by transforming a plant cell with an expression cassette that expresses a polynucleotide that inhibits the expression of the CNR polypeptide. The polynucleotide may inhibit the expression of the CNR polypeptide directly, by preventing transcription or translation of the CNR messenger RNA, or indirectly, by encoding a polypeptide that inhibits the transcription or translation of a CNR gene encoding a CNR polypeptide. Methods for inhibiting or eliminating the expression of a gene in a plant are well known in the art, and any such method may be used in the present invention to inhibit the expression of a CNR polypeptide.

In accordance with the present invention, the expression of a CNR polypeptide is inhibited if the protein level of the CNR polypeptide is less than 70% of the protein level of the same CNR polypeptide in a plant that has not been genetically modified or mutagenized to inhibit the expression of that CNR polypeptide. In particular embodiments of the invention, the protein level of the CNR polypeptide in a modified plant according to the invention is less than 60%, less than 50%, less than 40%, less than 30%, less than 20%, less than 10%, less than 5% or less than 2% of the protein level of the same CNR polypeptide in a plant that is not a mutant or that has not been genetically modified to inhibit the expression of that CNR polypeptide. The expression level of the CNR polypeptide may be measured directly, for example, by assaying for the level of CNR polypeptide expressed in the plant cell or plant, or indirectly, for example, by measuring the cell number regulator activity of the CNR polypeptide in the plant cell or plant, or by measuring the cell number in the plant. Methods for performing such assays are described elsewhere herein.

In other embodiments of the invention, the activity of the CNR polypeptides is reduced or eliminated by transforming a plant cell with an expression cassette comprising a polynucleotide encoding a polypeptide that inhibits the activity of a CNR polypeptide. The cell number regulator activity of a CNR polypeptide is inhibited according to the present invention if the cell number regulator activity of the CNR polypeptide is less than 70% of the cell number regulator activity of the same CNR polypeptide in a plant that has not been modified to inhibit the cell number regulator activity of that CNR polypeptide. In particular embodiments of the invention, the cell number regulator activity of the CNR polypeptide in a modified plant according to the invention is less than 60%, less than 50%, less than 40%, less than 30%, less than 20%, less than 10% or less than 5% of the cell number regulator activity of the same CNR polypeptide in a plant that that has not been modified to inhibit the expression of that CNR polypeptide. The cell number regulator activity of a CNR polypeptide is “eliminated” according to the invention when it is not detectable by the assay methods described elsewhere herein. Methods of determining the cell number regulator activity of a CNR polypeptide are described elsewhere herein.

In other embodiments, the activity of a CNR polypeptide may be reduced or eliminated by disrupting the gene encoding the CNR polypeptide. The invention encompasses mutagenized plants that carry mutations in CNR genes, where the mutations reduce expression of the CNR gene or inhibit the cell number regulator activity of the encoded CNR polypeptide.

Thus, many methods may be used to reduce or eliminate the activity of a CNR polypeptide. In addition, more than one method may be used to reduce the activity of a single CNR polypeptide. Non-limiting examples of methods of reducing or eliminating the expression of CNR polypeptides are given below.

1. Polynucleotide-Based Methods:

In some embodiments of the present invention, a plant is transformed with an expression cassette that is capable of expressing a polynucleotide that inhibits the expression of a CNR polypeptide of the invention. The term “expression” as used herein refers to the biosynthesis of a gene product, including the transcription and/or translation of said gene product. For example, for the purposes of the present invention, an expression cassette capable of expressing a polynucleotide that inhibits the expression of at least one CNR polypeptide is an expression cassette capable of producing an RNA molecule that inhibits the transcription and/or translation of at least one CNR polypeptide of the invention. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a CNR polypeptide are given below.

i. Sense Suppression/Cosuppression

In some embodiments of the invention, inhibition of the expression of a CNR polypeptide may be obtained by sense suppression or cosuppression. For cosuppression, an expression cassette is designed to express an RNA molecule corresponding to all or part of a messenger RNA encoding a CNR polypeptide in the “sense” orientation. Over expression of the RNA molecule can result in reduced expression of the native gene. Accordingly, multiple plant lines transformed with the cosuppression expression cassette are screened to identify those that show the greatest inhibition of CNR polypeptide expression.

The polynucleotide used for cosuppression may correspond to all or part of the sequence encoding the CNR polypeptide, all or part of the 5′ and/or 3′ untranslated region of a CNR polypeptide transcript, or all or part of both the coding sequence and the untranslated regions of a transcript encoding a CNR polypeptide. In some embodiments where the polynucleotide comprises all or part of the coding region for the CNR polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin, et al., (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell, et al., (1994) Proc. Natl. Acad. Sci. USA 91:3490-3496; Jorgensen, et al., (1996) Plant Mol. Biol. 31:957-973; Johansen and Carrington, (2001) Plant Physiol. 126:930-938; Broin, et al., (2002) Plant Cell 14:1417-1432; Stoutjesdijk, et al., (2002) Plant Physiol. 129:1723-1731; Yu, et al., (2003) Phytochemistry 63:753-763 and U.S. Pat. Nos. 5,034,323, 5,283,184 and 5,942,657; each of which is herein incorporated by reference. The efficiency of cosuppression may be increased by including a poly-dT region in the expression cassette at a position 3′ to the sense sequence and 5′ of the polyadenylation signal. See, US Patent Application Publication Number 2002/0048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

ii. Antisense Suppression

In some embodiments of the invention, inhibition of the expression of the CNR polypeptide may be obtained by antisense suppression. For antisense suppression, the expression cassette is designed to express an RNA molecule complementary to all or part of a messenger RNA encoding the CNR polypeptide. Over expression of the antisense RNA molecule can result in reduced expression of the native gene. Accordingly, multiple plant lines transformed with the antisense suppression expression cassette are screened to identify those that show the greatest inhibition of CNR polypeptide expression.

The polynucleotide for use in antisense suppression may correspond to all or part of the complement of the sequence encoding the CNR polypeptide, all or part of the complement of the 5′ and/or 3′ untranslated region of the CNR transcript, or all or part of the complement of both the coding sequence and the untranslated regions of a transcript encoding the CNR polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550 or greater may be used. Methods for using antisense suppression to inhibit the expression of endogenous genes in plants are described, for example, in Liu, et al., (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, US Patent Application Publication Number 2002/0048814, herein incorporated by reference.

iii. Double-Stranded RNA Interference

In some embodiments of the invention, inhibition of the expression of a CNR polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of CNR polypeptide expression. Methods for using dsRNA interference to inhibit the expression of endogenous plant genes are described in Waterhouse, et al., (1998) Proc. Natl. Acad. Sci. USA 95:13959-13964, Liu, et al., (2002) Plant Physiol. 129:1732-1743 and WO 1999/49029, WO 1999/53050, WO 1999/61631 and WO 2000/49035, each of which is herein incorporated by reference.

iv. Hairpin RNA Interference and Intron-Containing Hairpin RNA Interference

In some embodiments of the invention, inhibition of the expression of a CNR polypeptide may be obtained by hairpin RNA (hpRNA) interference or intron-containing hairpin RNA (ihpRNA) interference. These methods are highly efficient at inhibiting the expression of endogenous genes. See, Waterhouse and Helliwell, (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Alternatively, the base-paired stem region may correspond to a portion of a promoter sequence controlling expression of the gene to be inhibited. Thus, the base-paired stem region of the molecule generally determines the specificity of the RNA interference. hpRNA molecules are highly efficient at inhibiting the expression of endogenous genes, and the RNA interference they induce is inherited by subsequent generations of plants. See, for example, Chuang and Meyerowitz, (2000) Proc. Natl. Acad. Sci. USA 97:4985-4990; Stoutjesdijk, et al., (2002) Plant Physiol. 129:1723-1731 and Waterhouse and Helliwell, (2003) Nat. Rev. Genet. 4:29-38. Methods for using hpRNA interference to inhibit or silence the expression of genes are described, for example, in Chuang and Meyerowitz, (2000) Proc. Natl. Acad. Sci. USA 97:4985-4990; Stoutjesdijk, et al., (2002) Plant Physiol. 129:1723-1731; Waterhouse and Helliwell, (2003) Nat. Rev. Genet. 4:29-38; Pandolfini, et al., BMC Biotechnology 3:7 and US Patent Application Publication Number 2003/0175965; each of which is herein incorporated by reference. A transient assay for the efficiency of hpRNA constructs to silence gene expression in vivo has been described by Panstruga, et al., (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith, et al., (2000) Nature 407:319-320. In fact, Smith, et al., show 100% suppression of endogenous gene expression using ihpRNA-mediated interference. Methods for using ihpRNA interference to inhibit the expression of endogenous plant genes are described, for example, in Smith, et al., (2000) Nature 407:319-320; Wesley, et al., (2001) Plant J. 27:581-590; Wang and Waterhouse, (2001) Curr. Opin. Plant Biol. 5:146-150; Waterhouse and Helliwell, (2003) Nat. Rev. Genet. 4:29-38; Helliwell and Waterhouse, (2003) Methods 30:289-295 and US Patent Application Publication Number 2003/0180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 2002/00904, Mette, et al., (2000) EMBO J 19:5194-5201; Matzke, et al., (2001) Curr. Opin. Genet. Devel. 11:221-227; Scheid, et al., (2002) Proc. Natl. Acad. Sci., USA 99:13659-13662; Aufsaftz, et al., (2002) Proc. Nat'l. Acad. Sci. 99(4):16499-16506; Sijen, et al., (2001) Curr. Biol. 11:436-440), herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the CNR polypeptide). Methods of using amplicons to inhibit the expression of endogenous plant genes are described, for example, in Angell and Baulcombe, (1997) EMBO J. 16:3675-3684, Angell and Baulcombe, (1999) Plant J. 20:357-362 and U.S. Pat. No. 6,646,805, each of which is herein incorporated by reference.

vi. Ribozymes

In some embodiments, the polynucleotide expressed by the expression cassette of the invention is catalytic RNA or has ribozyme activity specific for the messenger RNA of the CNR polypeptide. Thus, the polynucleotide causes the degradation of the endogenous messenger RNA, resulting in reduced expression of the CNR polypeptide. This method is described, for example, in U.S. Pat. No. 4,987,071, herein incorporated by reference.

vii. Small Interfering RNA or Micro RNA

In some embodiments of the invention, inhibition of the expression of a CNR polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier, et al., (2003) Nature 425:257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of CNR expression, the 22-nucleotide sequence is selected from a CNR transcript sequence and contains 22 nucleotides of said CNR sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

2. Polypeptide-Based Inhibition of Gene Expression

In one embodiment, the polynucleotide encodes a zinc finger protein that binds to a gene encoding a CNR polypeptide, resulting in reduced expression of the gene. In particular embodiments, the zinc finger protein binds to a regulatory region of a CNR gene. In other embodiments, the zinc finger protein binds to a messenger RNA encoding a CNR polypeptide and prevents its translation. Methods of selecting sites for targeting by zinc finger proteins have been described, for example, in U.S. Pat. No. 6,453,242 and methods for using zinc finger proteins to inhibit the expression of genes in plants are described, for example, in US Patent Application Publication Number 2003/0037355; each of which is herein incorporated by reference.

3. Polypeptide-Based Inhibition of Protein Activity

In some embodiments of the invention, the polynucleotide encodes an antibody that binds to at least one CNR polypeptide, and reduces the cell number regulator activity of the CNR polypeptide. In another embodiment, the binding of the antibody results in increased turnover of the antibody-CNR complex by cellular quality control mechanisms. The expression of antibodies in plant cells and the inhibition of molecular pathways by expression and binding of antibodies to proteins in plant cells are well known in the art. See, for example, Conrad and Sonnewald, (2003) Nature Biotech. 21:35-36, incorporated herein by reference.

4. Gene Disruption

In some embodiments of the present invention, the activity of a CNR polypeptide is reduced or eliminated by disrupting the gene encoding the CNR polypeptide. The gene encoding the CNR polypeptide may be disrupted by any method known in the art. For example, in one embodiment, the gene is disrupted by transposon tagging. In another embodiment, the gene is disrupted by mutagenizing plants using random or targeted mutagenesis and selecting for plants that have reduced cell number regulator activity.

i. Transposon Tagging

In one embodiment of the invention, transposon tagging is used to reduce or eliminate the CNR activity of one or more CNR polypeptide. Transposon tagging comprises inserting a transposon within an endogenous CNR gene to reduce or eliminate expression of the CNR polypeptide. “CNR gene” is intended to mean the gene that encodes a CNR polypeptide according to the invention.

In this embodiment, the expression of one or more CNR polypeptide is reduced or eliminated by inserting a transposon within a regulatory region or coding region of the gene encoding the CNR polypeptide. A transposon that is within an exon, intron, 5′ or 3′ untranslated sequence, a promoter or any other regulatory sequence of a CNR gene may be used to reduce or eliminate the expression and/or activity of the encoded CNR polypeptide.

Methods for the transposon tagging of specific genes in plants are well known in the art. See, for example, Maes, et al., (1999) Trends Plant Sci. 4:90-96; Dharmapuri and Sonti, (1999) FEMS Microbiol. Lett. 179:53-59; Meissner, et al., (2000) Plant J. 22:265-274; Phogat, et al., (2000) J. Biosci. 25:57-63; Walbot, (2000) Curr. Opin. Plant Biol. 2:103-107; Gai, et al., (2000) Nucleic Acids Res. 28:94-96; Fitzmaurice, et al., (1999) Genetics 153:1919-1928). In addition, the TUSC process for selecting Mu insertions in selected genes has been described in Bensen, et al., (1995) Plant Cell 7:75-84; Mena, et al., (1996) Science 274:1537-1540 and U.S. Pat. No. 5,962,764; each of which is herein incorporated by reference.

ii. Mutant Plants with Reduced Activity

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see, Ohshima, et al., (1998) Virology 243:472-481; Okubara, et al., (1994) Genetics 137:867-874 and Quesada, et al., (2000) Genetics 154:421-436; each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See, McCallum, et al., (2000) Nat. Biotechnol. 18:455-457, herein incorporated by reference.

Mutations that impact gene expression or that interfere with the function (cell number regulator activity) of the encoded protein are well known in the art. Insertional mutations in gene exons usually result in null-mutants. Mutations in conserved residues are particularly effective in inhibiting the cell number regulator activity of the encoded protein. Conserved residues of plant CNR polypeptides suitable for mutagenesis with the goal to eliminate cell number regulator activity have been described. Such mutants can be isolated according to well-known procedures, and mutations in different CNR loci can be stacked by genetic crossing. See, for example, Gruis, et al., (2002) Plant Cell 14:2863-2882.

In another embodiment of this invention, dominant mutants can be used to trigger RNA silencing due to gene inversion and recombination of a duplicated gene locus. See, for example, Kusaba, et al., (2003) Plant Cell 15:1455-1467.

The invention encompasses additional methods for reducing or eliminating the activity of one or more CNR polypeptide. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972 and 5,871,984; each of which are herein incorporated by reference. See also, WO 1998/49350, WO 1999/07865, WO 1999/25821 and Beetham, et al., (1999) Proc. Natl. Acad. Sci. USA 96:8774-8778; each of which is herein incorporated by reference.

iii. Modulating Cell Number Regulator Activity

In specific methods, the level and/or activity of a cell number regulator in a plant is decreased by increasing the level or activity of the CNR polypeptide in the plant. Methods for increasing the level and/or activity of CNR polypeptides in a plant are discussed elsewhere herein. Briefly, such methods comprise providing a CNR polypeptide of the invention to a plant and thereby increasing the level and/or activity of the CNR polypeptide. In other embodiments, a CNR nucleotide sequence encoding a CNR polypeptide can be provided by introducing into the plant a polynucleotide comprising a CNR nucleotide sequence of the invention, expressing the CNR sequence, increasing the activity of the CNR polypeptide and thereby decreasing the number of tissue cells in the plant or plant part. In other embodiments, the CNR nucleotide construct introduced into the plant is stably incorporated into the genome of the plant.

In other methods, the number of cells in a plant tissue is increased by decreasing the level and/or activity of the CNR polypeptide in the plant. Such methods are disclosed in detail elsewhere herein. In one such method, a CNR nucleotide sequence is introduced into the plant and expression of said CNR nucleotide sequence decreases the activity of the CNR polypeptide, and thereby increasing the cell number in the plant or plant part. In other embodiments, the CNR nucleotide construct introduced into the plant is stably incorporated into the genome of the plant.

As discussed above, one of skill will recognize the appropriate promoter to use to modulate the level/activity of a cell number regulator in the plant. Exemplary promoters for this embodiment have been disclosed elsewhere herein.

Accordingly, the present invention further provides plants having a modified number of cells when compared to the number of cells of a control plant tissue. In one embodiment, the plant of the invention has an increased level/activity of the CNR polypeptide of the invention and thus has a decreased number of cells in the plant tissue. In other embodiments, the plant of the invention has a reduced or eliminated level of the CNR polypeptide of the invention and thus has an increased number of cells in the plant tissue. In other embodiments, such plants have stably incorporated into their genome a nucleic acid molecule comprising a CNR nucleotide sequence of the invention operably linked to a promoter that drives expression in the plant cell.

iv. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development or radial expansion.

Methods for modulating root development in a plant are provided. The methods comprise modulating the level and/or activity of the CNR polypeptide in the plant. In one method, a CNR sequence of the invention is provided to the plant. In another method, the CNR nucleotide sequence is provided by introducing into the plant a polynucleotide comprising a CNR nucleotide sequence of the invention, expressing the CNR sequence, and thereby modifying root development. In still other methods, the CNR nucleotide construct introduced into the plant is stably incorporated into the genome of the plant.

In other methods, root development is modulated by altering the level or activity of the CNR polypeptide in the plant. A decrease in CNR activity can result in at least one or more of the following alterations to root development, including, but not limited to, larger root meristems, increased in root growth, enhanced radial expansion, an enhanced vasculature system, increased root branching, more adventitious roots and/or an increase in fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, US Patent Application Publication Number 2003/0074698 and Werner, et al., (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by decreasing the activity and/or level of the CNR polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse (environmental) conditions. This trait relates to the size, depth and morphology of the root system. In addition, stimulating root growth and increasing root mass by decreasing the level and/or activity of the CNR polypeptide also finds use in promoting in vitro propagation of explants.

Furthermore, higher root biomass production due to an decreased level and/or activity of CNR activity has a direct effect on the yield and an indirect effect of production of compounds produced by root cells or transgenic root cells or cell cultures of said transgenic root cells. One example of an interesting compound produced in root cultures is shikonin, the yield of which can be advantageously enhanced by said methods.

Accordingly, the present invention further provides plants having modulated root development when compared to the root development of a control plant. In some embodiments, the plant of the invention has an increased level/activity of the CNR polypeptide of the invention and has enhanced root growth and/or root biomass. In other embodiments, such plants have stably incorporated into their genome a nucleic acid molecule comprising a CNR nucleotide sequence of the invention operably linked to a promoter that drives expression in the plant cell.

v. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length and leaf senescence. As used herein, “leaf development” and “shoot development” encompasses all aspects of growth of the different parts that make up the leaf system and the shoot system, respectively, at different stages of their development, both in monocotyledonous and dicotyledonous plants. Methods for measuring such developmental alterations in the shoot and leaf system are known in the art. See, for example, Werner, et al., (2001) PNAS 98:10487-10492 and US Patent Application Publication Number 2003/0074698, each of which is herein incorporated by reference.

The method for modulating shoot and/or leaf development in a plant comprises modulating the activity and/or level of a CNR polypeptide of the invention. In one embodiment, a CNR sequence of the invention is provided. In other embodiments, the CNR nucleotide sequence can be provided by introducing into the plant a polynucleotide comprising a CNR nucleotide sequence of the invention, expressing the CNR sequence, and thereby modifying shoot and/or leaf development. In other embodiments, the CNR nucleotide construct introduced into the plant is stably incorporated into the genome of the plant.

In specific embodiments, shoot or leaf development is modulated by increasing the level and/or activity of the CNR polypeptide in the plant. An increase in CNR activity can result in at least one or more of the following alterations in shoot and/or leaf development, including, but not limited to, reduced leaf number, reduced leaf surface, reduced vascular, shorter internodes and stunted growth and retarded leaf senescence, when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters and leaf-preferred promoters. Exemplary promoters have been disclosed elsewhere herein.

Increasing CNR activity and/or level in a plant results in shorter internodes and stunted growth. Thus, the methods of the invention find use in producing dwarf plants. In addition, as discussed above, modulation CNR activity in the plant modulates both root and shoot growth. Thus, the present invention further provides methods for altering the root/shoot ratio. Shoot or leaf development can further be modulated by decreasing the level and/or activity of the CNR polypeptide in the plant.

Accordingly, the present invention further provides plants having modulated shoot and/or leaf development when compared to a control plant. In some embodiments, the plant of the invention has an increased level/activity of the CNR polypeptide of the invention. In other embodiments, the plant of the invention has a decreased level/activity of the CNR polypeptide of the invention.

vi Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the CNR polypeptide has not been modulated. “Modulating floral development” further includes any alteration in the timing of the development of a plant's reproductive tissue (i.e., a delayed or a accelerated timing of floral development) when compared to a control plant in which the activity or level of the CNR polypeptide has not been modulated. Macroscopic alterations may include changes in size, shape, number or location of reproductive organs, the developmental time period that these structures form, or the ability to maintain or proceed through the flowering process in times of environmental stress. Microscopic alterations may include changes to the types or shapes of cells that make up the reproductive organs.

The method for modulating floral development in a plant comprises modulating CNR activity in a plant. In one method, a CNR sequence of the invention is provided. A CNR nucleotide sequence can be provided by introducing into the plant a polynucleotide comprising a CNR nucleotide sequence of the invention, expressing the CNR sequence, and thereby modifying floral development. In other embodiments, the CNR nucleotide construct introduced into the plant is stably incorporated into the genome of the plant.

In specific methods, floral development is modulated by increasing the level or activity of the CNR polypeptide in the plant. An increase in CNR activity can result in at least one or more of the following alterations in floral development, including, but not limited to, retarded flowering, reduced number of flowers, partial male sterility and reduced seed set, when compared to a control plant. Inducing delayed flowering or inhibiting flowering can be used to enhance yield in forage crops such as alfalfa. Methods for measuring such developmental alterations in floral development are known in the art. See, for example, Mouradov, et al., (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters and inflorescence-preferred promoters.

In other methods, floral development is modulated by decreasing the level and/or activity of the CNR sequence of the invention. Such methods can comprise introducing a CNR nucleotide sequence into the plant and decreasing the activity of the CNR polypeptide. In other methods, the CNR nucleotide construct introduced into the plant is stably incorporated into the genome of the plant. Decreasing expression of the CNR sequence of the invention can modulate floral development during periods of stress. Such methods are described elsewhere herein. Accordingly, the present invention further provides plants having modulated floral development when compared to the floral development of a control plant. Compositions include plants having a decreased level/activity of the CNR polypeptide of the invention and having an altered floral development. Compositions also include plants having a decreased level/activity of the CNR polypeptide of the invention wherein the plant maintains or proceeds through the flowering process in times of stress.

Methods are also provided for the use of the CNR sequences of the invention to increase seed size and/or weight. The method comprises increasing the activity of the CNR sequences in a plant or plant part, such as the seed. An increase in seed size and/or weight comprises an increased size or weight of the seed and/or an increase in the size or weight of one or more seed part including, for example, the embryo, endosperm, seed coat, aleurone or cotyledon.

As discussed above, one of skill will recognize the appropriate promoter to use to increase seed size and/or seed weight. Exemplary promoters of this embodiment include constitutive promoters, inducible promoters, seed-preferred promoters, embryo-preferred promoters and endosperm-preferred promoters.

The method for decreasing seed size and/or seed weight in a plant comprises increasing CNR activity in the plant. In one embodiment, the CNR nucleotide sequence can be provided by introducing into the plant a polynucleotide comprising a CNR nucleotide sequence of the invention, expressing the CNR sequence, and thereby decreasing seed weight and/or size. In other embodiments, the CNR nucleotide construct introduced into the plant is stably incorporated into the genome of the plant.

It is further recognized that increasing seed size and/or weight can also be accompanied by an increase in the speed of growth of seedlings or an increase in early vigor. As used herein, the term “early vigor” refers to the ability of a plant to grow rapidly during early development, and relates to the successful establishment, after germination, of a well-developed root system and a well-developed photosynthetic apparatus. In addition, an increase in seed size and/or weight can also result in an increase in plant yield when compared to a control.

Accordingly, the present invention further provides plants having an increased seed weight and/or seed size when compared to a control plant. In other embodiments, plants having an increased vigor and plant yield are also provided. In some embodiments, the plant of the invention has a decreased level/activity of the CNR polypeptide of the invention and has an increased seed weight and/or seed size. In other embodiments, such plants have stably incorporated into their genome a nucleic acid molecule comprising a CNR nucleotide sequence of the invention operably linked to a promoter that drives expression in the plant cell.

vii. Method of Use for CNR Promoter Polynucleotides

The polynucleotides comprising the CNR promoters disclosed in the present invention, as well as variants and fragments thereof, are useful in the genetic manipulation of any host cell, preferably plant cell, when assembled with a DNA construct such that the promoter sequence is operably linked to a nucleotide sequence comprising a polynucleotide of interest. In this manner, the CNR promoter polynucleotides of the invention are provided in expression cassettes along with a polynucleotide sequence of interest for expression in the host cell of interest. As discussed in Example 2 below, the CNR promoter sequences of the invention are expressed in a variety of tissues and thus the promoter sequences can find use in regulating the temporal and/or the spatial expression of polynucleotides of interest.

Synthetic hybrid promoter regions are known in the art. Such regions comprise upstream promoter elements of one polynucleotide operably linked to the promoter element of another polynucleotide. In an embodiment of the invention, heterologous sequence expression is controlled by a synthetic hybrid promoter comprising the CNR promoter sequences of the invention, or a variant or fragment thereof, operably linked to upstream promoter element(s) from a heterologous promoter. Upstream promoter elements that are involved in the plant defense system have been identified and may be used to generate a synthetic promoter. See, for example, Rushton, et al., (1998) Curr. Opin. Plant Biol. 1:311-315. Alternatively, a synthetic CNR promoter sequence may comprise duplications of the upstream promoter elements found within the CNR promoter sequences.

It is recognized that the promoter sequence of the invention may be used with its native CNR coding sequences. A DNA construct comprising the CNR promoter operably linked with its native CNR gene may be used to transform any plant of interest to bring about a desired phenotypic change, such as modulating cell number, modulating root, shoot, leaf, floral and embryo development, stress tolerance and any other phenotype described elsewhere herein.

The promoter nucleotide sequences and methods disclosed herein are useful in regulating expression of any heterologous nucleotide sequence in a host plant in order to vary the phenotype of a plant. Various changes in phenotype are of interest including modifying the fatty acid composition in a plant, altering the amino acid content of a plant, altering a plant's pathogen defense mechanism and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics and commercial products. Genes of interest include, generally, those involved in oil, starch, carbohydrate or nutrient metabolism as well as those affecting kernel size, sucrose loading and the like.

In certain embodiments the nucleic acid sequences of the present invention can be used in combination (“stacked”) with other polynucleotide sequences of interest in order to create plants with a desired phenotype. The combinations generated can include multiple copies of any one or more of the polynucleotides of interest. The polynucleotides of the present invention may be stacked with any gene or combination of genes to produce plants with a variety of desired trait combinations, including but not limited to traits desirable for animal feed such as high oil genes (e.g., U.S. Pat. No. 6,232,529); balanced amino acids (e.g., hordothionins (U.S. Pat. Nos. 5,990,389; 5,885,801; 5,885,802 and 5,703,409); barley high lysine (Williamson, et al., (1987) Eur. J. Biochem. 165:99-106; and WO 98/20122) and high methionine proteins (Pedersen, et al., (1986) J. Biol. Chem. 261:6279; Kirihara, et al., (1988) Gene 71:359 and Musumura, et al., (1989) Plant Mol. Biol. 12: 123)); increased digestibility (e.g., modified storage proteins (U.S. patent application Ser. No. 10/053,410, filed Nov. 7, 2001) and thioredoxins (U.S. patent application Ser. No. 10/005,429, filed Dec. 3, 2001)), the disclosures of which are herein incorporated by reference. The polynucleotides of the present invention can also be stacked with traits desirable for insect, disease or herbicide resistance (e.g., Bacillus thuringiensis toxic proteins (U.S. Pat. Nos. 5,366,892; 5,747,450; 5,737,514; 5723,756; 5,593,881; Geiser, et al., (1986) Gene 48:109); lectins (Van Damme, et al., (1994) Plant Mol. Biol. 24:825); fumonisin detoxification genes (U.S. Pat. No. 5,792,931); avirulence and disease resistance genes (Jones, et al., (1994) Science 266:789; Martin, et al., (1993) Science 262:1432; Mindrinos, et al., (1994) Cell 78:1089); acetolactate synthase (ALS) mutants that lead to herbicide resistance such as the S4 and/or Hra mutations; inhibitors of glutamine synthase such as phosphinothricin or basta (e.g., bar gene); and glyphosate resistance (EPSPS gene)); and traits desirable for processing or process products such as high oil (e.g., U.S. Pat. No. 6,232,529); modified oils (e.g., fatty acid desaturase genes (U.S. Pat. No. 5,952,544; WO 1994/11516)); modified starches (e.g., ADPG pyrophosphorylases (AGPase), starch synthases (SS), starch branching enzymes (SBE) and starch debranching enzymes (SDBE)); and polymers or bioplastics (e.g., U.S. Pat. No. 5,602,321; beta-ketothiolase, polyhydroxybutyrate synthase, and acetoacetyl-CoA reductase (Schubert, et al., (1988) J. Bacteriol. 170:5837-5847) facilitate expression of polyhydroxyalkanoates (PHAs)), the disclosures of which are herein incorporated by reference. One could also combine the polynucleotides of the present invention with polynucleotides affecting agronomic traits such as male sterility (e.g., see, U.S. Pat. No. 5,583,210), stalk strength, flowering time, or transformation technology traits such as cell cycle regulation or gene targeting (e.g., WO 1999/61619; WO 2000/17364; WO 1999/25821), the disclosures of which are herein incorporated by reference.

In one embodiment, sequences of interest improve plant growth and/or crop yields. For example, sequences of interest include agronomically important genes that result in improved primary or lateral root systems. Such genes include, but are not limited to, nutrient/water transporters and growth induces. Examples of such genes, include but are not limited to, maize plasma membrane H⁺-ATPase (MHA2) (Frias, et al., (1996) Plant Cell 8:1533-44); AKT1, a component of the potassium uptake apparatus in Arabidopsis, (Spalding, et al., (1999) J Gen Physiol 113:909-18); RML genes which activate cell division cycle in the root apical cells (Cheng, et al., (1995) Plant Physiol 108:881); maize glutamine synthetase genes (Sukanya, et al., (1994) Plant Mol Biol 26:1935-46) and hemoglobin (Duff, et al., (1997) J. Biol. Chem. 27:16749-16752, Arredondo-Peter, et al., (1997) Plant Physiol. 115:1259-1266; Arredondo-Peter, et al., (1997) Plant Physiol 114:493-500 and references sited therein). The sequence of interest may also be useful in expressing antisense nucleotide sequences of genes that that negatively affects root development.

Additional, agronomically important traits such as oil, starch and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802 and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson, et al., (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. patent application Ser. No. 08/740,682, filed Nov. 1, 1996, and WO 1998/20133, the disclosures of which are herein incorporated by reference. Other proteins include methionine-rich plant proteins such as from sunflower seed (Lilley, et al., (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen, et al., (1986) J. Biol. Chem. 261:6279; Kirihara, et al., (1988) Gene 71:359; both of which are herein incorporated by reference) and rice (Musumura, et al., (1989) Plant Mol. Biol. 12:123, herein incorporated by reference). Other agronomically important genes encode latex, Floury 2, growth factors, seed storage factors and transcription factors.

Insect resistance genes may encode resistance to pests that have great yield drag such as rootworm, cutworm, European Corn Borer and the like. Such genes include, for example, Bacillus thuringiensis toxic protein genes (U.S. Pat. Nos. 5,366,892; 5,747,450; 5,736,514; 5,723,756; 5,593,881 and Geiser, et al., (1986) Gene 48:109) and the like.

Genes encoding disease resistance traits include detoxification genes, such as against fumonosin (U.S. Pat. No. 5,792,931); avirulence (avr) and disease resistance (R) genes (Jones, et al., (1994) Science 266:789; Martin, et al., (1993) Science 262:1432; and Mindrinos, et al., (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids and levels of cellulose. In corn, modified hordothionin proteins are described in U.S. Pat. Nos. 5,703,049, 5,885,801, 5,885,802 and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see, Schubert, et al., (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones and the like. The level of proteins, particularly modified proteins having improved amino acid distribution to improve the nutrient value of the plant, can be increased. This is achieved by the expression of such proteins having enhanced amino acid content.

This invention can be better understood by reference to the following non-limiting examples. It will be appreciated by those skilled in the art that other embodiments of the invention may be practiced without departing from the spirit and the scope of the invention as herein disclosed and claimed.

### EXAMPLES

### Example 1

### Isolation of CNR sequences

A routine for identifying all members of a gene family was employed to search for the CNR genes of interest. A diverse set of all the known members of the gene family as protein sequences was prepared. This data includes sequences from other species. These species are searched against a proprietary maize sequence dataset and a nonredundant set of overlapping hits is identified. Separately, one takes the nucleotide sequences of any genes of interest in hand and searches against the database and a nonredundant set of all overlapping hits are retrieved. The set of protein hits are then compared to the nucleotide hits. If the gene family is complete, all of the protein hits are contained within the nucleotide hits. The CNR family of genes consists of 12 members.

### Example 2

### CNR Sequence Analysis

The CNR polypeptides of the current invention have common characteristics with tomato fw2.2 polypeptide (SEQ ID NO: 45). The relationship between the genes of the invention and the tomato fw2.2 is shown in Table 2.

TABLE 2

| | % Identity | % Similarity |
|---|---|---|
| Gene | to Fw2-2 | to Fw2-2 |
| ZmCNR02 | 53.59 | 62.98 |
| ZmCNR10 | 49.69 | 62.58 |
| ZmCNR09 | 47.43 | 58.29 |
| ZmCNR03 | 46.71 | 59.28 |
| ZmCNR01 | 46.6 | 57.59 |
| ZmCNR07 | 46.11 | 58.89 |
| ZmCNR04 | 41.1 | 55.83 |
| ZmCNR11 | 41.1 | 55.21 |
| ZmCNR12 | 36.2 | 49.08 |
| ZmCNR05 | 33.7 | 48.91 |
| ZmCNR08 | 27.9 | 35.19 |
| ZmCNR06 | 21.34 | 30.54 |

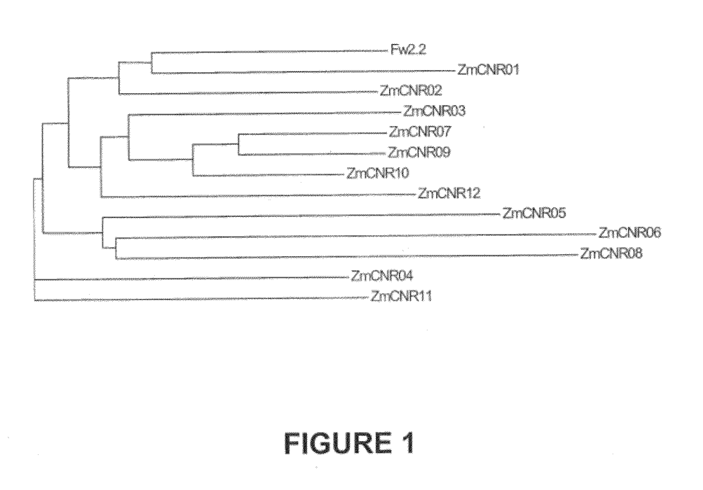

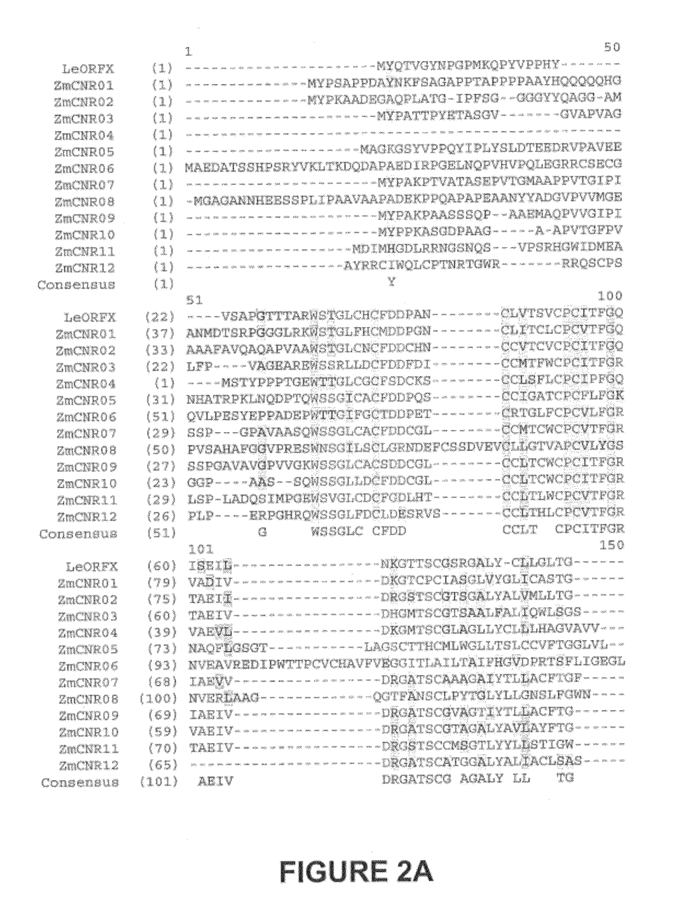

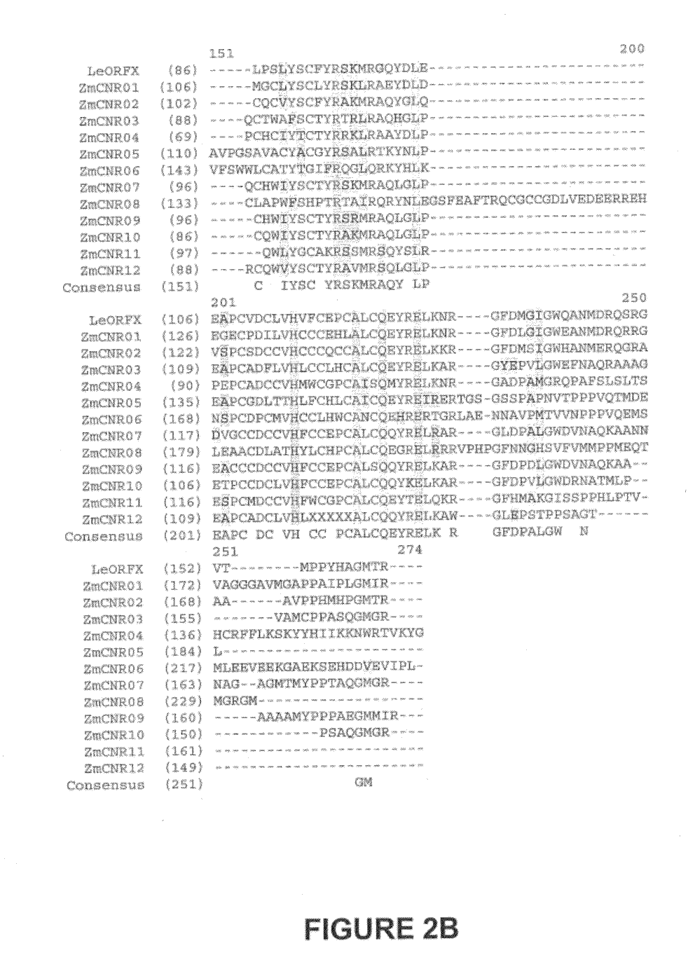

In addition, a clustal dendrogram and alignment of the Tomato fw2.2 sequence (SEQ ID NO: 2) with the 12 maize gene translations is provided in FIGS. 1 and 2 respectively.

### Example 3

### CNR Expression Patterns in Maize using MPSS

MPSS stands for Massively Parallel Signature Sequencing, a technique invented and commercialized by Lynx Therapeutics, Inc. of Hayward, Calif. MPSS and related technologies have been described in publications by Brenner, et al., (Nature Biotechnol. (2000) 18:630-634 and PNAS (2000) 97:1665-1670). Like SAGE (Serial Analysis of Gene Expression), MPSS produces short sequence signatures produced from a defined position within an mRNA, and the relative abundance of these signatures in a given library represents a quantitative estimate of expression of that gene. The MPSS signatures are 17 bp in length, and can uniquely identify >95% of all genes in Arabidopsis.

The CNR sequences were matched to MPSS data, and matching tags (GATC-17mers) were curated. Ideally, the correct tag for a gene is in the plus strand proximal to but just up from the poly A tail, and it is gene specific. Where more than one tag matches a gene, one will usually choose the one closest to the poly A tail, which is also usually the one with the highest gene expression. Where the tag matches more than one gene, the correct gene association is usually the one that has an EST distribution that best corresponds to the expression pattern revealed by the MPSS data.

Expression of the various CNR sequences revealed that:

ZmCNR 1 was weakly expressed in various tissues—most consistently in stalk and tassel spikelets

ZmCNR 2 was expressed more strongly in various tissues, and appears to be silk preferred

ZmCNR 3 is the only member of the group which demonstrated strong pollen preferred expression

ZmCNR 4 expression was not detectable

ZmCNR 5 was weakly expressed in various tissues

ZmCNR 6 was strongly expressed in many tissues—but not pollen—expressed rather abundantly in seed tissues

ZmCNR 7 and 9 were expressed in various tissues, but were silk preferred

ZmCNR 8 was expressed moderately in various tissues, with a bias toward tassel spikelets.

### Specific Tissue Expression Data Relating to ZmCNR02

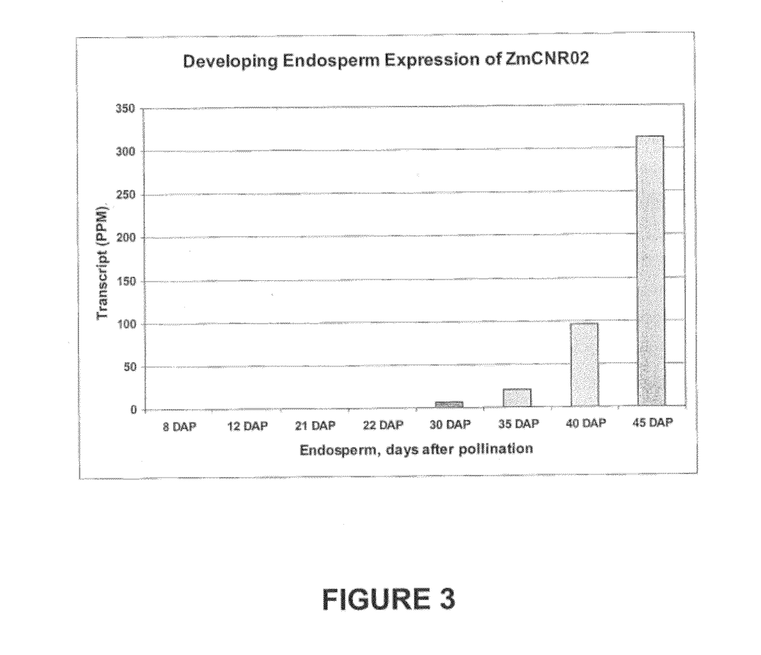

Endosperm Development—The pattern of ZmCNR02 gene expression as revealed by MPSS data reveals that the gene expression is very low in the early stages of endosperm development (in early days after pollination—DAP), but that as the endosperm matures (higher DAP), the expression of ZmCNR02 increases as illustrated in FIG. 3. Thus this pattern of expression in endosperm is consistent with a role of ZmCNR02 in negatively regulating cell number.

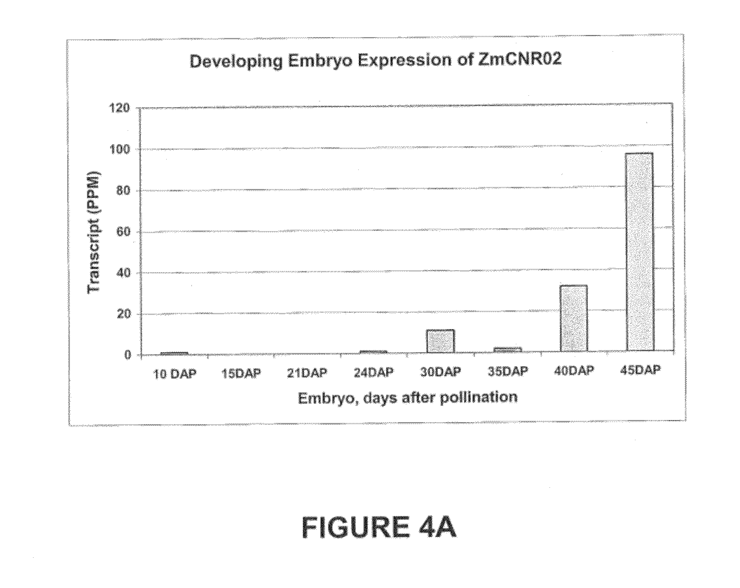

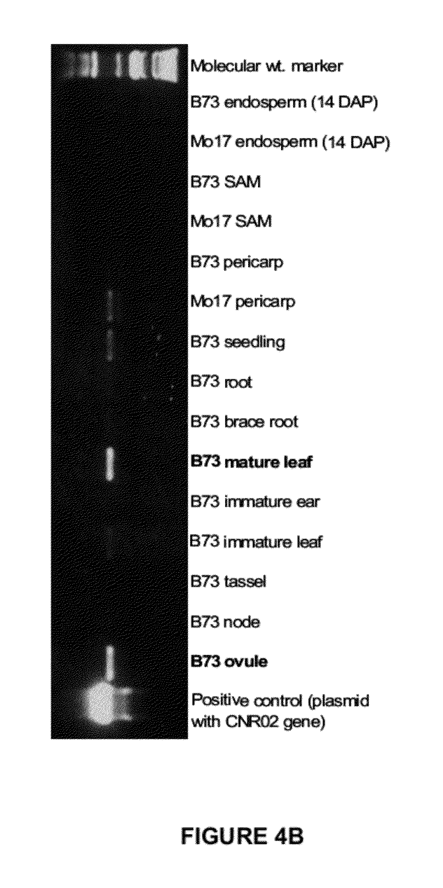

Embryo Development—The seed embryo development is scored in terms of days after pollination (DAP). The pattern of ZmCNR02 expression rises towards the end of embryo development after 30 DAP, with the highest expression at 45 DAP, see, FIG. 4A. This corresponds to the period of completion of cell number growth, this pattern of expression is consistent with a role for ZmCNR02 as a negative cell number regulator.

Ovule Development—FIG. 4B illustrates results from 35 cycles of RT-PCR performed with different maize tissues, including endosperm (14 DAP), shoot apical meristem, pericarp, seedling, root, brace root, mature and immature leaf, immature ear, immature tassel, node and ovule. Consistent with the Lynx MPSS profiling data, the expression of this gene is detected mostly in the tissue where there is little growth activity, such as mature leaf. Interestingly, a very high expression is detected in the ovule tissue. The ovule (pre-fertilization) has no cell division activity and is at a rest stage. ZmCNR02 is expressed at a very high level in the ovule, comparable to the level in the mature leaf tissue. However, immediately after fertilization when active cell division begins, the ZmCNR02 expression is dramatically reduced to a minimal level, as shown in the early embryo and endosperm development (see, expression demonstrated in FIGS. 3 and 4A).

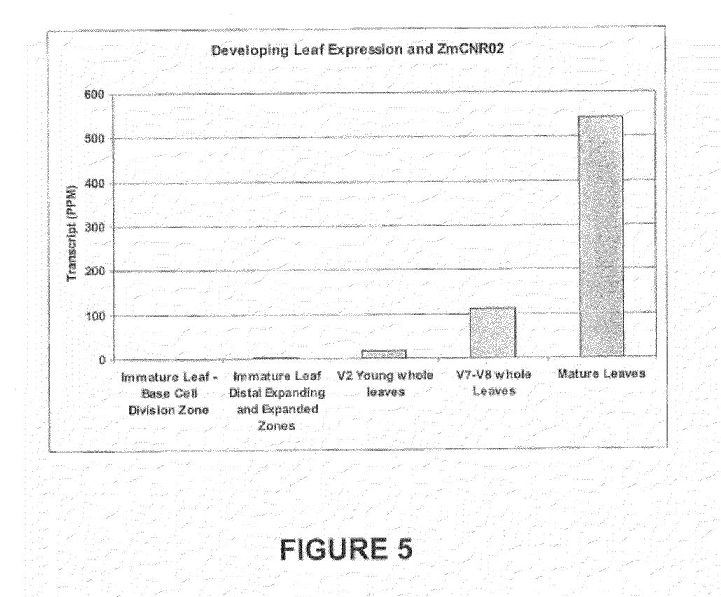

Leaf Development—Several samples were assayed in relation to developing maize leaves as shown in FIG. 5. The basal region of immature leaves, the region of most active cell division, showed no ZmCNR02 expression. The distal expanding and expanded portions of the same immature leaves showed a small but noticeable ZmCNR02 expression. A series of whole leaves from young plants (V2) to middle stage leaves (V7-V8) to mature leaves, showed progressively higher ZmCNR02 expression. This expression pattern is consistent with ZmCNR02 being related to negative control of cell number; its expression is highest in leaf stages that are undergoing little cell division.

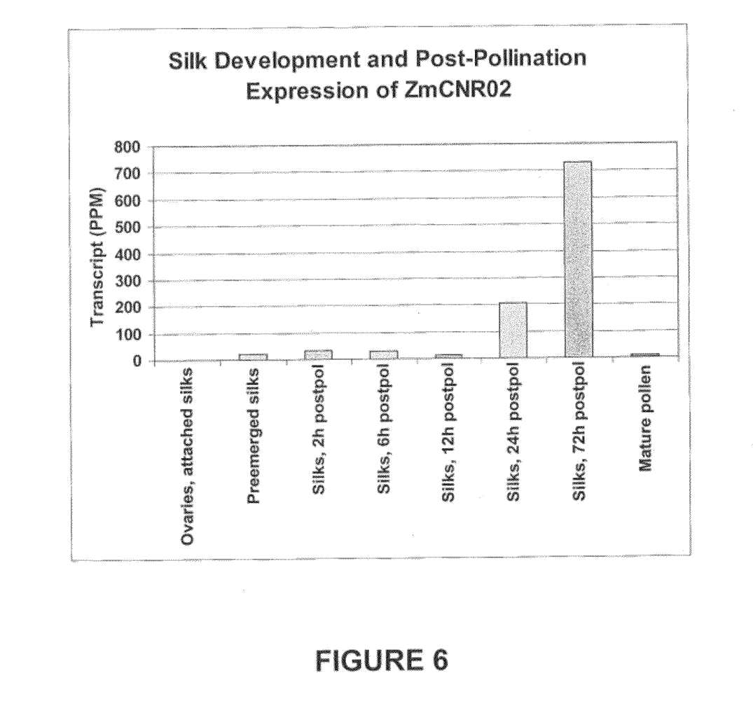

Carpels, Silk Development, and Pollen—The silks, ovary walls and pericarp are analogous to the dicot flower carpel. ZmCNR02 expression is detected in the latter two. The ZmCNR02 expression is in the maize ‘carpels’ by virtue of the silk and pericarp expression. The pericarp samples assayed are fairly late in development and are compromised by remaining endosperm tissue. The silk tissues are fairly easy to gather and assay for gene expression. In the young growing silks (those still attached to the ovaries) the expression of ZmCNR02 expression is not detected. Then moving through a series of pre-emergent to post emergent silks, and thence through a post pollination series, the expression of ZmCNR02 increases. For comparison the pollen sample is offered indicating that the increase of ZmCNR02 expression is not derived from the pollen landing on the silks. As silks mature, and especially after they are pollinated, the cell division slows and stops. The pattern of ZmCNR02 expression in silks (a carpel tissue) is consistent with a negative cell number regulator, see, FIG. 6.

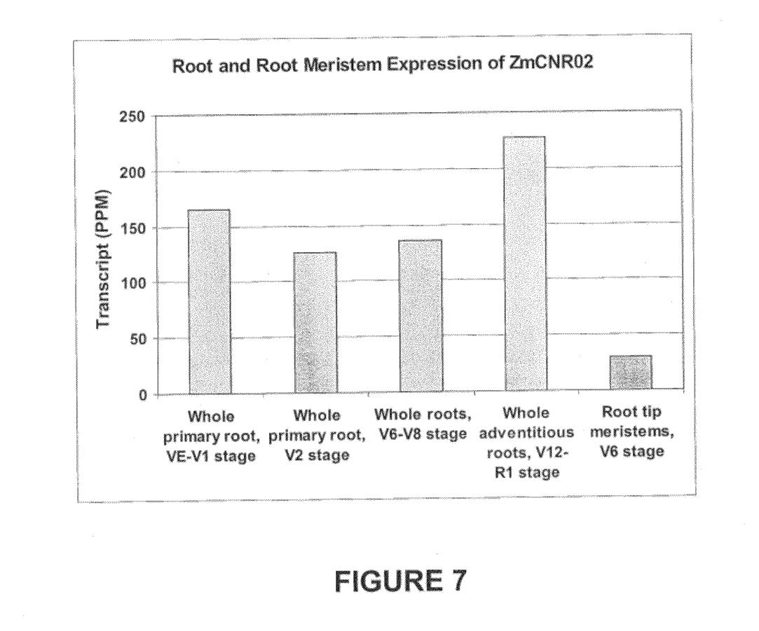

Root and Root Meristems—A comparison of whole roots (with meristems) to root tips (meristem enriched), as presented in FIG. 7, shows that ZmCNR02 expression is higher in whole roots than root tips. The ZmCNR02 expression having higher expression in areas of the root not actively dividing, and the expression pattern in roots is consistent with as a negative regulator of cell number (division).

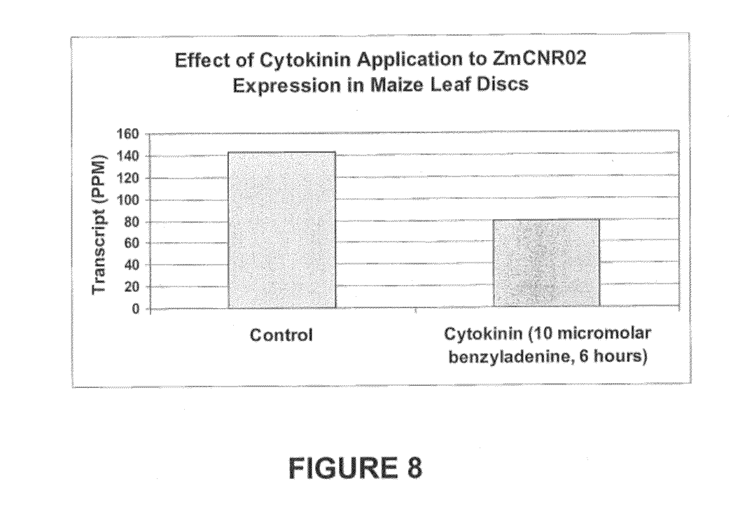

Cytokinin Treatment—Data from an experiment showing that the ZmCNR02 genes' expression, as revealed by MPSS transcript assay, decreases in excised maize leaf discs, when 10 micromolar benzyladenine is added for 6 hours and is shown in FIG. 8. The experimental and control samples used:
Corn leaf disc, Ctrl—[leaf discs from ear leaf (cerca L9) of R1 plants, discs 5 mm diameter. Cultured 6 hours at 25° C.]
Corn leaf disc, +BA—[leaf discs from ear leaf (cerca L9) of R1 plants, discs 5 mm diameter. Cultured 6 hours at 25° C. in 10 microMol Benzyladenine]

This result offers additional evidence that the expression of ZmCNR02 is consistent with a role in negatively regulating cell number. The addition of a plant hormone that is known to induce cell number (cell division) results in the DECLINE in expression of ZmCNR02, as expected per the hypothesis that this gene negatively regulates cell number.

### Example 4

### Transformation and Regeneration of Transgenic Plants

Immature maize embryos from greenhouse donor plants are bombarded with a plasmid containing the CNR sequence operably linked to the drought-inducible promoter RAB17 promoter (Vilardell, et al., (1990) Plant Mol Biol 14:423-432) and the selectable marker gene PAT, which confers resistance to the herbicide Bialaphos. Alternatively, the selectable marker gene is provided on a separate plasmid. Transformation is performed as follows. Media recipes follow below.

Preparation of Target Tissue:

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA:

A plasmid vector comprising the CNR sequence operably linked to an ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

Particle Gun Treatment:

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment:

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for increased drought tolerance. Assays to measure improved drought tolerance are routine in the art and include, for example, increased kernel-earring capacity yields under drought conditions when compared to control maize plants under identical environmental conditions. Alternatively, the transformed plants can be monitored for a modulation in meristem development (i.e., a decrease in spikelet formation on the ear). See, for example, Bruce, et al., (2002) Journal of Experimental Botany 53:1-13.

Bombardment and Culture Media:

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O) and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog, (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/1 myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 5

### Agrobacterium-Mediated Transformation

For Agrobacterium-mediated transformation of maize with an antisense sequence of the CNR sequence of the present invention, preferably the method of Zhao is employed (U.S. Pat. No. 5,981,840, and PCT Patent Publication Number WO 1998/32326; the contents of which are hereby incorporated by reference). Briefly, immature embryos are isolated from maize and the embryos contacted with a suspension of Agrobacterium, where the bacteria are capable of transferring the antisense CNR sequences to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants. Plants are monitored and scored for a modulation in meristem development. For instance, alterations of size and appearance of the shoot and floral meristems and/or increased yields of leaves, flowers, and/or fruits.

### Example 6

### Analysis of ZmCNR02 Expression in Maize Leaf Tissue

Collections of maize leaf section series by growth activity:

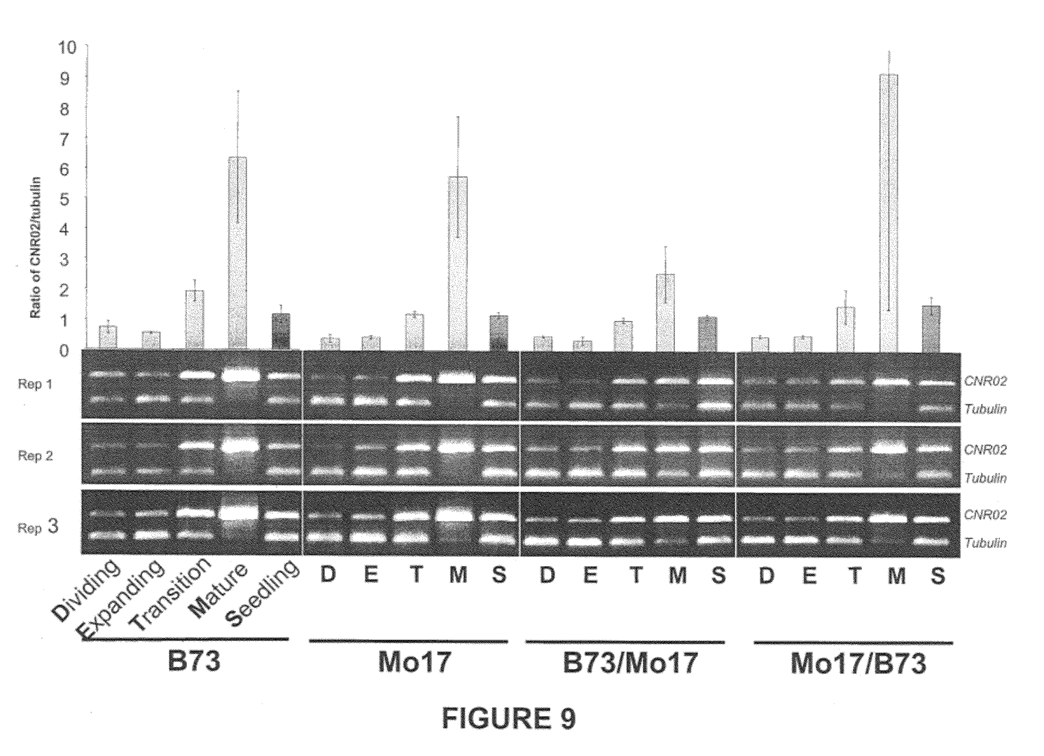

Leaf sections of different growth activity are collected from seedlings at V3 stage. The leaf blades of 1^{st}, 2^{nd}, 3^{rd} leaves that are fully opened are collected and pooled as the mature leaf tissue. The sheath of these 3 leaves are removed and discarded. The remaining whorl tissue (mostly leaf tissue) is then sectioned from the base to top as:
1. 0-6 mm: mostly cell dividing zone (including shoot apical meristem)
2. 6-20 mm: mostly cell expanding zone
3. 20 mm-tip: transition zone
4. Mature leaf: the fully opened leaf blades as described above with little growth activity
5. The whole seedling that has mixture of growing and mature leaf tissues
   The growth activity of these tissues is in the order as 1>2>3>4. #5 is a mixture. FIG. 9 shows the RT-PCR analysis of ZmCNR02 multiplexing with tubulin as a control. There are two main points from this data:
1. The expression of ZmCNR02 (shown as a ratio of ZmCNR02/tubulin) is negatively correlated with the growth activity, increasing from sample #1 to #4.
2. This trend is consistent seen in all four genotypes, including inbreds and their reciprocal hybrids B73, Mo17, B73/Mo17 and Mo17/B73.

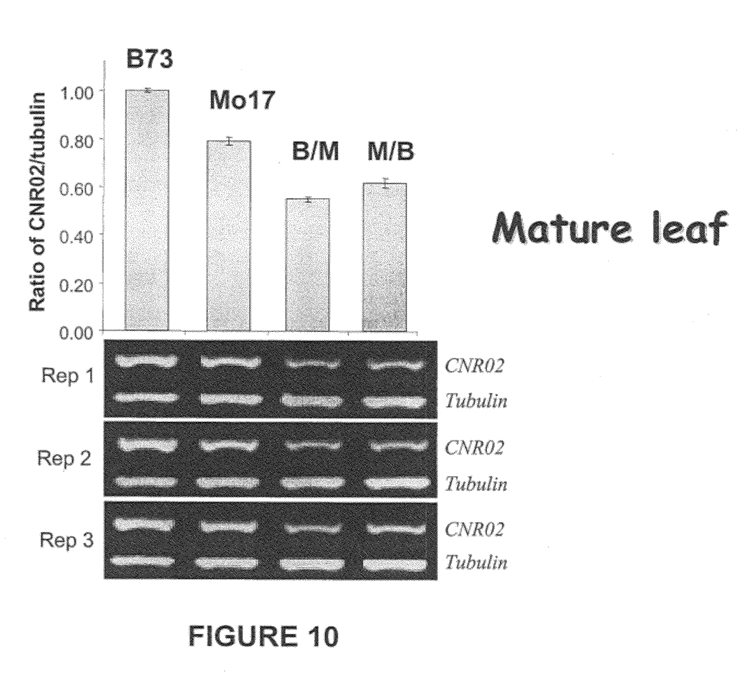

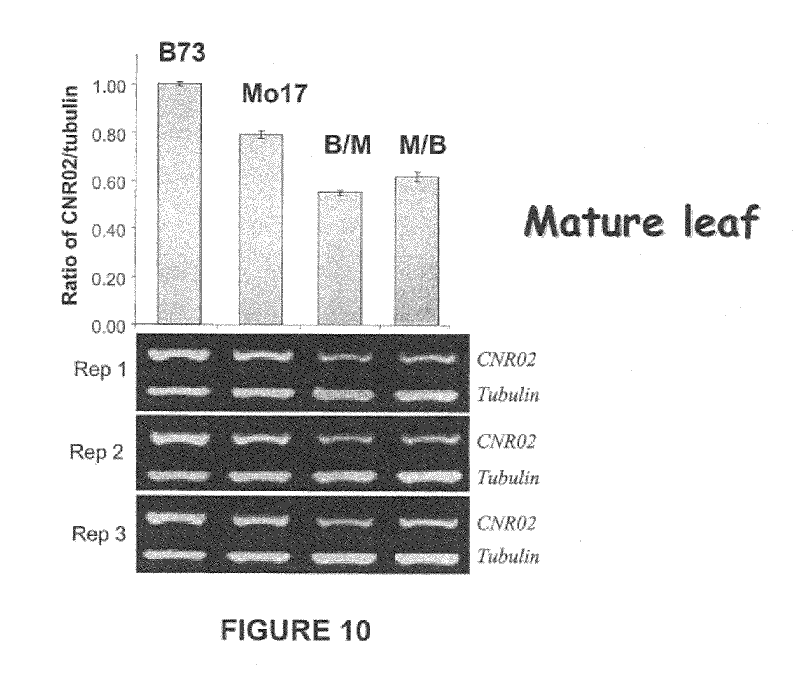

The FIG. 10 is a repeated RT-PCR assay with the mature leaf tissue, where we had to modify the PCR protocol to increase the amplification of tubulin that was out-competed by ZmCNR02's high expression. The figure shows well that the expression level of ZmCNR02 in both hybrids is significantly higher than the inbred parents, which is consistent with that hybrids grow faster and are more vigorous than inbreds.

### Example 7

### Soybean Embryo Transformation

Soybean embryos are bombarded with a plasmid containing an antisense CNR sequences operably linked to an ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein, et al., (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell, et al., (1985) Nature 313:810-812), the hygromycin phosphotransferase gene from plasmid pJR225 (from E. coli; Gritz, et al., (1983) Gene 25:179-188), and the 3′ region of the nopaline synthase gene from the T-DNA of the Ti plasmid of Agrobacterium tumefaciens. The expression cassette comprising an antisense CNR sequence operably linked to the ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 8

### Sunflower Meristem Tissue Transformation

Sunflower meristem tissues are transformed with an expression cassette containing an antisense CNR sequences operably linked to a ubiquitin promoter as follows (see also, EP Patent Number 0 486233, herein incorporated by reference, and Malone-Schoneberg, et al., (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer, et al., (Schrammeijer, et al., (1990) Plant Cell Rep. 9:55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige, et al., (1962) Physiol. Plant., 15:473-497), Shepard's vitamin additions (Shepard, (1980) in Emergent Techniques for the Genetic Improvement of Crops (University of Minnesota Press, St. Paul, Minn.), 40 mg/l adenine sulfate, 30 g/l sucrose, 0.5 mg/l 6-benzyl-aminopurine (BAP), 0.25 mg/l indole-3-acetic acid (IAA), 0.1 mg/l gibberellic acid (GA3), pH 5.6, and 8 g/l Phytagar.

The explants are subjected to microprojectile bombardment prior to Agrobacterium treatment (Bidney, et al., (1992) Plant Mol. Biol. 18:301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS1000® particle acceleration device.

Disarmed Agrobacterium tumefaciens strain EHA105 is used in all transformation experiments. A binary plasmid vector comprising the expression cassette that contains the CNR gene operably linked to the ubiquitin promoter is introduced into Agrobacterium strain EHA105 via freeze-thawing as described by Holsters, et al., (1978) Mol. Gen. Genet. 163:181-187. This plasmid further comprises a kanamycin selectable marker gene (i.e, nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for a modulation in meristem development (i.e., an alteration of size and appearance of shoot and floral meristems).

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by CNR activity analysis of leaf extracts while transgenic seeds harvested from NPTII-positive T₀ plants are identified by CNR activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/l 6-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it is dropped on the center of the surface of macrocarrier. Each plate is bombarded twice with 650 psi rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD₆₀₀. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for a modulation in meristem development (i.e., an alteration of size and appearance of shoot and floral meristems). After positive (i.e., a decrease in CNR expression) explants are identified, those shoots that fail to exhibit a decrease in CNR activity are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate protein activity assay. At this time, the positive shoots recovered from a single node will generally have been enriched in the transgenic sector detected in the initial assay prior to nodal culture.

Recovered shoots positive for a decreased CNR expression are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

### Example 9

### Variants of CNR Sequences

A. Variant Nucleotide Sequences of CNR That Do Not Alter the Encoded Amino Acid Sequence

The CNR nucleotide sequences are used to generate variant nucleotide sequences having the nucleotide sequence of the open reading frame with about 70%, 75%, 80%, 85%, 90% and 95% nucleotide sequence identity when compared to the starting unaltered ORF nucleotide sequence of the corresponding SEQ ID NO. These functional variants are generated using a standard codon table. While the nucleotide sequence of the variants are altered, the amino acid sequence encoded by the open reading frames do not change.

B. Variant Amino Acid Sequences of CNR Polypeptides

Variant amino acid sequences of the CNR polypeptides are generated. In this example, one amino acid is altered. Specifically, the open reading frames are reviewed to determine the appropriate amino acid alteration. The selection of the amino acid to change is made by consulting the protein alignment (with the other orthologs and other gene family members from various species). An amino acid is selected that is deemed not to be under high selection pressure (not highly conserved) and which is rather easily substituted by an amino acid with similar chemical characteristics (i.e., similar functional side-chain). Using the protein alignment set forth in FIG. 2, an appropriate amino acid can be changed. Once the targeted amino acid is identified, the procedure outlined in the following section C is followed. Variants having about 70%, 75%, 80%, 85%, 90% and 95% nucleic acid sequence identity are generated using this method.

C. Additional Variant Amino Acid Sequences of CNR Polypeptides

In this example, artificial protein sequences are created having 80%, 85%, 90% and 95% identity relative to the reference protein sequence. This latter effort requires identifying conserved and variable regions from the alignment set forth in FIG. 2 and then the judicious application of an amino acid substitutions table. These parts will be discussed in more detail below.

Largely, the determination of which amino acid sequences are altered is made based on the conserved regions among CNR protein or among the other CNR polypeptides. Based on the sequence alignment, the various regions of the CNR polypeptide that can likely be altered are represented in lower case letters, while the conserved regions are represented by capital letters. It is recognized that conservative substitutions can be made in the conserved regions below without altering function. In addition, one of skill will understand that functional variants of the CNR sequence of the invention can have minor non-conserved amino acid alterations in the conserved domain.

Artificial protein sequences are then created that are different from the original in the intervals of 80-85%, 85-90%, 90-95%, and 95-100% identity. Midpoints of these intervals are targeted, with liberal latitude of plus or minus 1%, for example. The amino acids substitutions will be effected by a custom Perl script. The substitution table is provided below in Table 3.

TABLE 3
Substitution Table

| | Strongly | | |
|---|---|---|---|
| | Similar and | Rank of | |
| | Optimal | Order to | |
| Amino Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the CNR polypeptides are generating having about 80%, 85%, 90% and 95% amino acid identity to the starting unaltered ORF nucleotide sequence of SEQ ID NO: 3, 6, 10 or 14.

### Example 10

### Transgenic Maize Plants

25 T₀ transgenic maize plants containing the ZmCNR01 construct under the control of the ubiquitin promoter were generated. These transgenic plants were grown in greenhouse conditions. Each of the 25 plants was found to have suppressed growth throughout their development. The extent of the growth suppression correlated with the copy number of the transgene. Transgenic plants with higher copy number had corresponding reductions in plant growth. Transgenic maize plants having one, two and four copies of the transgene, show approximately 30-50%, 60-70% and 80-90% reduction in plant height, respectively. These transgenic plants also contained reduced organ and tissue size, including smaller tassels, ears and leaves. The reduction in growth of organs and tissues may be associated with reduced cell number.

### Example 11

### Transgenic Maize Callus

Transgenic maize callus tissue expressing the ZmCNR02 gene under the control of the ubiquitin promoter exhibited significantly inhibited growth in cell (callus) culture. Individual plants associated with these calli would be expected to have a reduction in plant size and/or reduced organ and tissue size. Further evaluation of transgenic plants would present a more uniform genetic background for comparison. ZmCNR01 callus did not reveal the same characteristic reduction in callus growth, but the transgenic plants did. The differences in expression of the two genes may be related to protein action strength or tissue-response to the gene function.

Together the results for ZmCNR01 and ZmCNR02 indicate that these genes are capable of negatively regulating maize tissue growth. While the general presumed function of the tomato FW2.2, namely negative cell number regulation, and associated reduction in tissue size, is apparently preserved for these maize genes, these experiments demonstrate that cell number/tissue size control is exhibited in a very different plant and plant architecture, and in diverse tissues that are distinct from the specific instance of the tomato fruit carpels. Accordingly, this information argues that ZmCNR01 and ZmCNR02, and likely other maize ZmCNR genes, could be used to control tissue growth as exemplified.

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

What is claimed is:

1. An isolated polynucleotide selected from the group consisting of:
a. a polynucleotide having at least 70% sequence identity, as determined by the GAP algorithm under default parameters, to the full length sequence of a polynucleotide selected from the group consisting of SEQ ID NOS: 1, 3, 5, 7, 9, 11, 13, 15, 17, 19, 21 and 23; wherein the polynucleotide encodes a polypeptide that functions as a modifier of cell number;
b. a polynucleotide encoding a polypeptide selected from the group consisting of SEQ ID NO: 2, 4, 6, 8, 10, 12, 14, 16, 18, 20, 22 and 24;
c. a polynucleotide selected from the group consisting of SEQ ID NOS: 1, 3, 5, 7, 9, 11, 13, 15, 17, 19, 21 and 23 and
d. a polynucleotide which is complementary to the polynucleotide of (a), (b) or (c).

2. A recombinant expression cassette, comprising the polynucleotide of claim 1, wherein the polynucleotide is operably linked, in sense or anti-sense orientation, to a promoter.

3. A host cell comprising the expression cassette of claim 2.

4. A transgenic plant comprising the recombinant expression cassette of claim 2.

5. The transgenic plant of claim 4, wherein said plant is a monocot.

6. The transgenic plant of claim 4, wherein said plant is a dicot.

7. The transgenic plant of claim 4, wherein said plant is selected from the group consisting of: maize, soybean, sunflower, sorghum, canola, wheat, alfalfa, cotton, rice, barley, millet, peanut and cocoa.

8. A transgenic seed from the transgenic plant of claim 4.

9. A method of modulating the cell number in plants, comprising:
a. introducing into a plant cell a recombinant expression cassette comprising the polynucleotide of claim 1 operably linked to a promoter; and
b. culturing the plant under plant cell growing conditions; wherein the cell number in said plant cell is modulated.

10. The method of claim 9, wherein the plant cell is from a plant selected from the group consisting of: maize, soybean, sunflower, sorghum, canola, wheat, alfalfa, cotton, rice, barley, millet, peanut and cocoa.

11. A method of modulating the cell number in a plant, comprising:
a. introducing into a plant cell a recombinant expression cassette comprising the polynucleotide of claim 1 operably linked to a promoter;
b. culturing the plant cell under plant cell growing conditions; and
c. regenerating a plant form said plant cell; wherein the cell number in said plant is modulated.

12. The method of claim 11, wherein the plant is selected from the group consisting of: maize, soybean, sorghum, canola, wheat, alfalfa, cotton, rice, barley, millet, peanut, and cocoa.

13. A method of decreasing the cell number regulatory polypeptide activity in a plant cell, comprising:
a. providing a nucleotide sequence comprising at least 15 consecutive nucleotides of the complement of SEQ ID NO: 1, 3, 5, 7, 9, 11, 13, 15, 17, 19, 21 or 23;
b. providing a plant cell comprising an mRNA having the sequence set forth in SEQ ID NO: 1, 3, 5, 7, 9, 11, 13, 15, 17, 19, 21 or 23; and
c. introducing the nucleotide sequence of step (a) into the plant cell, wherein the nucleotide sequence inhibits expression of the mRNA in the plant cell.

14. The method of claim 13, wherein said plant cell is from a monocot.

15. The method of claim 14, wherein said monocot is maize, wheat, rice, barley, sorghum or rye.

16. The method of claim 13, wherein said plant cell is from a dicot.

17. The transgenic plant of claim 4, wherein the cell number regulatory activity in said plant is decreased.

18. The transgenic plant of claim 17, wherein the plant has enhanced root growth.

19. The transgenic plant of claim 17, wherein the plant has increased seed size.

20. The transgenic plant of claim 17, wherein the plant has increased seed weight.

21. The transgenic plant of claim 17, wherein the plant has seed with increased embryo size.

22. The transgenic plant of claim 17, wherein the plant has increased leaf size.

23. The transgenic plant of claim 17, wherein the plant has increased seedling vigor.

24. The transgenic plant of claim 17, wherein the plant has enhanced silk emergence.

25. The transgenic plant of claim 17, wherein the plant has increased ear size.

26. The transgenic plant of claim 4, wherein the cell number regulatory activity in said plant is increased.

27. The transgenic plant of claim 26, wherein the plant has decreased root growth.

28. The transgenic plant of claim 26, wherein the plant has decreased seed size.

29. The transgenic plant of claim 26, wherein the plant has decreased seed weight.

30. The transgenic plant of claim 26, wherein the plant has decreased embryo size.

31. The transgenic plant of claim 26, wherein the plant has decreased tassel production.


SEQUENCE LISTING (45 sequences)

SEQ ID NO: 1 - DNA, 910 bp, Zea mays
actcgagtcgagtcttgccgacctggggcctgctgcgcctggatgaagagctgtgctgagagaatcattaaagcagcaccgggtccttgctttgcgcgttgctgttcgcataaacaagagatcctagttctactcccaagcaatgtatccgtcagcccctccggacgcgtataacaagttcagcgccggggctccgccaacggcgccgccgccgccggcagcgtaccaccagcagcagcagcagcacggagcgaacatggacacttcacgccccggcggcgggctgaggaaatggtccaccggcctcttccactgcatggacgacccggggaactgtctcatcacatgcctgtgcccctgcgtcacgttcgggcaggtcgctgacatcgtggacaagggcacctgcccatgtatcgcgagcggactggtgtacgggctcatctgcgcgtcgacggggatggggtgcctgtactcgtgcctctaccggtccaagctgagggccgagtacgacctggacgaaggggagtgcccggacatcctggtgcactgctgctgcgagcacctggctctgtgccaagagtaccgcgagctcaagaaccgcggcttcgacctggggatcggctgggaggctaacatggaccggcagaggcgaggagttgccggcggcggcgcggtgatgggggcgccgccggccataccgctgggcatgattaggtagatgattgattgatgcggtggcagaacttgctttcgtggttcttttgtttgactatgtaatgtatcttcgtttccatcgattctatgtaatttaactgtccctttttttggctctctattgtggaggacgctgggtgtgaatagcactgttttggatgggtttgtgattggtctaaaaaaaaaaaaaaaaaaa

SEQ ID NO: 2 - protein, 191 aa, Zea mays
MYPSAPPDAYNKFSAGAPPTAPPPPAAYHQQQQQHGANMDTSRPGGGLRKWSTGLFHCMDDPGNCLITCLCPCVTFGQVADIVDKGTCPCIASGLVYGLICASTGMGCLYSCLYRSKLRAEYDLDEGECPDILVHCCCEHLALCQEYRELKNRGFDLGIGWEANMDRQRRGVAGGGAVMGAPPAIPLGMIR

SEQ ID NO: 3 - DNA, 813 bp, Zea mays
gcacaaggcaataaataaaaagcccaacgctgggctccgctagctctcggtctctctggagtctggactcgcctccacggctccaccatgtatcccaaggcggcagacgaaggtgcgcagccgctggccacgggcatccccttcagcggcggcggcggctactaccaggcgggcggcgcgatggcggcggcgttcgcggtgcaggcgcaggcgcccgtcgccgcctggtccaccgggctctgcaactgcttcgacgactgccacaactgctgcgtgacgtgcgtgtgcccgtgcatcacgttcgggcagaccgcggagatcatcgaccggggctccacgtcctgcggcaccagcggggcgctgtacgcgctcgtcatgctgctcaccggctgtcagtgcgtctactcctgcttctaccgcgccaagatgcgcgcgcagtacggcctccaggtgagcccctgctccgactgctgcgtgcactgctgctgccagtgctgcgcgctctgccaggagtaccgcgagctcaagaagcgaggcttcgacatgagcataggatggcatgcgaacatggagaggcaggggcgcgccgccgccgccgtgccgccgcacatgcatcctgggatgacccgctgacgctctgccgctcgcctcacttctgctgaggaaatcaagtgatttggtattggtccgctcccagcaggcagtatcaactactgtaaccaatccatgatctgtatgcggtatcgggctgaactgatactttatggacttgttgcttcaaaaaaaaaaaaaaaaaaaaaaaaaaaaaaaaaa

SEQ ID NO: 4 - protein, 181 aa, Zea mays
MYPKAADEGAQPLATGIPFSGGGGYYQAGGAMAAAFAVQAQAPVAAWSTGLCNCFDDCHNCCVTCVCPCITFGQTAEIIDRGSTSCGTSGALYALVMLLTGCQCVYSCFYRAKMRAQYGLQVSPCSDCCVHCCCQCCALCQEYRELKKRGFDMSIGWHANMERQGRAAAAVPPHMHPGMTR

SEQ ID NO: 5 - DNA, 836 bp, Zea mays
cgaccacctgcgctgccgctgccgtgccaagtcgccgggcaagctccggatcggggcgcgcgcgcgcggggccgccgccgccgccattatattgggttattagttaggccagctggtcattgcatcggagagatgtatccggccactacgccctacgagacggcgtccggggtgggcgtggcgccggtggccggcttgttccccgtcgccggagaggccagggagtggtcgtcgaggctcctggactgcttcgacgacttcgacatctgctgcatgacgttttggtgcccgtgcatcacgttcgggcggacggcggagatcgtggaccacggcatgacgtcgtgcgggacgagcgcggcgctgttcgcgctgatccagtggctgtcggggtcgcagtgcacgtgggccttctcctgcacgtaccgcaccaggctgcgcgcgcagcacgggctccccgaggcgccctgcgccgacttcctcgtccacctctgctgcctccactgcgcgctctgccaggagtacagggagctaaaggcgcgcggctacgagcccgtcctcggctgggagttcaacgcccagagggccgccgccggcgtcgccatgtgcccgccggcctcgcaggggatggggcgctgatcatccgtcgaccagccagccagcccgtcggggtttgattcatgtctccgatctgttggttcatgcatgcatgtctccatttaattcatctgttcatatattcatgtctccagatccagacggtacatataataatatattcagttgttttacgtaaaatcgagaaaaaaaaaaaaaaaaaaaaaaaaaaaaaaaaaaaa

SEQ ID NO: 6 - protein, 167 aa, Zea mays
MYPATTPYETASGVGVAPVAGLFPVAGEAREWSSRLLDCFDDFDICCMTFWCPCITFGRTAEIVDHGMTSCGTSAALFALIQWLSGSQCTWAFSCTYRTRLRAQHGLPEAPCADFLVHLCCLHCALCQEYRELKARGYEPVLGWEFNAQRAAAGVAMCPPASQGMGR

SEQ ID NO: 7 - DNA, 527 bp, Zea mays
agtatcaattatcataacctccgactccgacgatctgaactgtgaagatgagcacttatccaccgccgacgggggaatggaccaccggcctctgtggctgcttcagcgactgcaagagctgttgcctgtcgttcttgtgcccgtgtatcccgttcggacaggtggcagaggtcttggacaagggcatgacatcctgtggtctggccggcctcctctactgtttgctgctgcatgccggggtggctgtggtcccgtgccactgcatctacacctgcacctaccgtcgcaagctccgggcggcgtacgacctgccgccggagccgtgcgccgattgctgcgtccacatgtggtgcgggccatgcgccatctcccagatgtaccgggagctcaagaacagaggcgccgacccggccatgggtaggcagccagccttctctctctctctcaccagtcactgccgtttctttctgaaatcaaaatattaccatatcataaaaaaaaattggcgcaccgttaaatacggataa

SEQ ID NO: 8 - protein, 159 aa, Zea mays
MSTYPPPTGEWTTGLCGCFSDCKSCCLSFLCPCIPFGQVAEVLDKGMTSCGLAGLLYCLLLHAGVAVVPCHCIYTCTYRRKLRAAYDLPPEPCADCCVHMWCGPCAISQMYRELKNRGADPAMGRQPAFSLSLTSHCRFFLKSKYYHIIKKNWRTVKYG

SEQ ID NO: 9 - DNA, 1009 bp, Zea mays
aattcctgcccgggcgggcgtagaggcgaagcattcgttggggacgaacgtttagggccggggatcggggaacacgaggctgcgcgttggcgcaagcaagcaaattttgggtcctagcttgaaggactgccatcttttttttttgggatggctggaaaaggaagctatgtacctccacaatatattcccttatacagcctagataccgaagaggatcgtgtccctgccgtggaagagaaccatgctacgcgccctaaactaaaccaggatccaacacaatggtcatctggcatctgtgcctgttttgatgacccccagagctgttgtattggtgcgacttgcccctgtttcctttttggaaagaatgcacaattcttgggatctggaactcttgctggatcatgcactacacattgcatgttgtggggccttctcacaagtctatgctgtgtatttactggaggtctagtattagcagttccaggatctgccgttgcttgttatgcttgcggatatcgcagtgcactaagaacaaagtacaatcttccggaagcaccctgtggcgatttgacgacacacttattctgtcacttgtgtgctatatgccaggagtacagggagatccgtgagagaacaggcagtggctcctcaccagctcctaatgtgactccacctccagttcagacgatggatgagctttgatctttaactgatatgaatgtatgttatctgacaaactttggtgtgtggcctggtggctagtgctgcctttgcctgttggcattttgacatatttttgttcttgggctctcacaacgctgctatgttgtttgtagagttgtaaacacgggttttgaactagtgaaccactgacagttcacggaagttgtaaacacagttgtaccatggaactgtagtagaattattaagtgtatttttttttggctatttatattggtgagaggatgggaagggaaaaaaaaaaaaaaaaaaaaaaaaaaaaaaaaaa

SEQ ID NO: 10 - protein, 184 aa, Zea mays
MAGKGSYVPPQYIPLYSLDTEEDRVPAVEENHATRPKLNQDPTQWSSGICACFDDPQSCCIGATCPCFLFGKNAQFLGSGTLAGSCTTHCMLWGLLTSLCCVFTGGLVLAVPGSAVACYACGYRSALRTKYNLPEAPCGDLTTHLFCHLCAICQEYREIRERTGSGSSPAPNVTPPPVQTMDEL

SEQ ID NO: 11 - DNA, 976 bp, Zea mays
gcaaggccggggtcggcagcagaggggagcagagagatctcattctccgatccgcccaggccaccatggcggaggatgctacgagcagccacccgtcacgctacgtcaagctcaccaaggaccaggacgcccccgccgaggacatccgccccggcgagctcaaccagccagttcacgtcccgcagctcgaaggccggaggtgtagcgagtgcggtcaggtcctgcccgagagctacgagccgcccgccgacgagccctggaccaccgggatcttcggatgcaccgatgacccagagacctgccgaactggattgttttgcccctgcgtgctgtttgggcgcaacgttgaggctgttagggaggacatcccttggacaactccttgcgtgtgccatgctgtattcgttgaaggagggatcacgctggcgattctgacggcgatatttcacggtgttgatccgaggacgtcattcctgattggagaaggtctggtgttcagctggtggttatgtgctacctacactggcatcttccgccaggggcttcagaggaaatatcatctcaagaactctccgtgtgacccatgcatggtccactgctgcttgcactggtgtgccaactgccaggagcatcgcgagaggacgggacggcttgcagagaacaacgcagtgcccatgacggttgtgaacccgcctccggtgcaagagatgagcatgctggaggaggtggaggagaagggagcagagaagagtgaacacgatgatgtggaggtcattcctctatagataatagggtcggagtccgagtgaggatagcactagcagcatcagatgacgatcacaacttctggtcattcctctatagttagttagttattgatctttcaacaacaaaatgtggtggtgtggcaactgtccatttttattatcccaaataataatactgtactacgttacggcccaaaaaaaaaaaaaaa

SEQ ID NO: 12 - protein, 239 aa, Zea mays
MAEDATSSHPSRYVKLTKDQDAPAEDIRPGELNQPVHVPQLEGRRCSECGQVLPESYEPPADEPWTTGIFGCTDDPETCRTGLFCPCVLFGRNVEAVREDIPWTTPCVCHAVFVEGGITLAILTAIFHGVDPRTSFLIGEGLVFSWWLCATYTGIFRQGLQRKYHLKNSPCDPCMVHCCLHWCANCQEHRERTGRLAENNAVPMTVVNPPPVQEMSMLEEVEEKGAEKSEHDDVEVIPL

SEQ ID NO: 13 - DNA, 801 bp, Zea mays
cggacgcgtggggtgaacgcgagagccgaccgtctcagtgtgcgcgcgaagaagagaaggaggaggaggaggaggaagaagaagaagaatgtatccggccaagcccaccgtcgcgaccgccagcgagccggtaaccgggatggcggcgccgccggtgaccggcatccccatcagcagccccggccccgccgtcgcggccagccagtggtcctcgggcctctgcgcctgcttcgacgactgcggcctctgctgcatgacgtgctggtgcccgtgcgtgacgttcgggcggatcgcggaggtcgtggaccgcggcgcgacgtcgtgcgcggccgcgggggccatctacacgctgctggcctgcttcacggggttccagtgccactggatctactcctgcacgtaccgctccaagatgcgcgcgcagctggggctccccgacgtcggctgctgcgactgctgcgtccacttctgctgcgagccgtgcgcgctctgccagcagtacagggagctcagggcacgcggcttggaccccgcgctcggctgggacgtcaacgcccagaaggcggccaataataacgccggcgccggcatgacgatgtacccgccgacggcgcaggggatgggccgctaattacgctaccagctccatcggtaaactcatatgttgttgtgttgtgtcagccagatcatcgcgtacgtacgggtatacttgtgatatgtaatgtcagggaccggcatgcccatgtgtttcaagatatttcacgttcaagtcctttctgtctgctcactatccgaatgaaa

SEQ ID NO: 14 - protein, 180 aa, Zea mays
MYPAKPTVATASEPVTGMAAPPVTGIPISSPGPAVAASQWSSGLCACFDDCGLCCMTCWCPCVTFGRIAEVVDRGATSCAAAGAIYTLLACFTGFQCHWIYSCTYRSKMRAQLGLPDVGCCDCCVHFCCEPCALCQQYRELRARGLDPALGWDVNAQKAANNNAGAGMTMYPPTAQGMGR

SEQ ID NO: 15 - DNA, 1103 bp, Zea mays
gtccccttccctcgcctcgcgttaggtagcctgcttccgacccgcacatctccctccggttctcgccatgggcgccggcgccaacaaccacgaggagtcgagccccctcatcccagctgcggttgccgcccccgcggacgagaagcccccgcaggctccggcgccggaggccgccaattactatgccgacggggtcccggtcgtgatgggcgagcccgtgtcggcccacgccttcggcggcgtcccgcgggagagctggaactccgggatcctctcctgcctcggccgcaacgacgagttctgcagcagcgacgtcgaagtgtgtcttcttggaacggtagcaccatgtgtgctctatggtagcaatgttgagaggcttgctgctggacaaggcacatttgcaaacagctgtttgccttacactgggctgtatttgcttgggaactctctctttgggtggaactgccttgccccatggttctctcatcccactcgtactgctattcgacaacgctacaatcttgagggtagctttgaggctttcaccaggcaatgtgggtgctgcggcgatctggtcgaggacgaggagaggcgtgagcacctagaggccgcctgtgaccttgcgacccactacctctgccacccctgcgccctctgccaggagggtcgcgagctgcgccgcagggttccccaccctggattcaacaatgggcactccgtctttgtcatgatgccgcccatggagcagaccatggggcgtggcatgtgagctattccaccaccttccctgccctagttttatctgtgcttcccgtggtttatcatctgctgctgttggccttgatgtgtgatgtgtcgtttgttcgtcagtaaatacgagtttgtgatatgaggtcgtgctgggaactttggattgtttgcttcttatcgactgcagtttggattctgaacagtcacttatctcctgtgttactgtgtttgtacggtggtcctcgatgggcctgtaaatgtgaaatgcatttcgtcgtaacttaggccccgtttcaaaaaaaaaaaaaaaaaaaaaaaaaaaaaaaaaaaaaaaaaaaaaaaaaaaaaaaaaa

SEQ ID NO: 16 - protein, 233 aa, Zea mays
MGAGANNHEESSPLIPAAVAAPADEKPPQAPAPEAANYYADGVPVVMGEPVSAHAFGGVPRESWNSGILSCLGRNDEFCSSDVEVCLLGTVAPCVLYGSNVERLAAGQGTFANSCLPYTGLYLLGNSLFGWNCLAPWFSHPTRTAIRQRYNLEGSFEAFTRQCGCCGDLVEDEERREHLEAACDLATHYLCHPCALCQEGRELRRRVPHPGFNNGHSVFVMMPPMEQTMGRGM

SEQ ID NO: 17 - DNA, 873 bp, Zeaq mays
gaattaagcgagcgccggccgaggcagacacaacccgcagcaacaacttgcatcggcacggacgaccgattgagagcctcggcgttcgtacgccacgccaccaagagactctgtgcaacaagtgaagggacgaaatgtatccggccaagcccgccgcctcctccagccagccagcggccgagatggcgcagccggtggtcggcatccccatcagcagccccggcgcggtcgcggtcgggccggtcgtcggcaagtggtcctccggcctctgcgcctgctccgacgactgcggcctctgctgcctgacgtgctggtgcccgtgcatcacgttcggccgcatcgcggagatcgtggaccgcggcgcgacgtcgtgcggcgtggcggggaccatctacacgctgctggcctgcttcacgggctgccactggatctactcctgcacgtaccgctccaggatgcgcgcgcagctcggcctccccgaagcctgctgctgcgactgctgcgtccacttctgctgcgagccctgcgcgctctcccagcagtacagggagctcaaggcccgcggattcgaccccgacctcggctgggacgtcaacgcgcagaaggccgccgccgccgccgccatgtacccgccgccggcggaggggatgatgatccgctaagctaagctagctacctcgtcgccgatccggcccaacctccaacgctaaactcagatgcgttgcgtcagtcagatcatcgcgtacgtacgtgttttgtacgtatggtcgtgtatatttgtacgtgatgcgtatatatataatgttatgactgaatgtctgagttttaagatttgtacgtttgaatccttttcttaaaaaaaaaaaaaaaaaa

SEQ ID NO: 18 - protein, 175 aa, Zea mays
MYPAKPAASSSQPAAEMAQPVVGIPISSPGAVAVGPVVGKWSSGLCACSDDCGLCCLTCWCPCITFGRIAEIVDRGATSCGVAGTIYTLLACFTGCHWIYSCTYRSRMRAQLGLPEACCCDCCVHFCCEPCALSQQYRELKARGFDPDLGWDVNAQKAAAAAAMYPPPAEGMMIR

SEQ ID NO: 19 - DNA, 752 bp, Zea mays
caaacgcaaccgcagctacagcacagcacagcacagcacaacacgtcggcatgtatccgcccaaggccagcggcgatccggccgccggggcggcgccggtgactggcttccccgtcggcgggcctgccgcctcctcccagtggtcctccggcctgttggactgcttcgacgactgcggcctctgctgcctgacgtgctggtgcccgtgcatcacgttcgggcgcgtggcggagatcgtggaccgcggcgcgacgtcgtgcggcacggcgggggcgctgtacgcggtgctggcctacttcacgggctgccagtggatctactcgtgcacgtaccgcgccaagatgcgcgcccagctcggcctccccgagaccccctgctgcgactgcctcgtccacttctgctgcgagccgtgcgcgctctgccagcagtacaaggagctcaaggcccgcggcttcgaccccgtcctcggctgggaccgcaacgccactatgctgcctccgtccgcacaggggatgggccgctgaccgctgaccggccagcctctgcgtaaataaataattaatgcttatatatgtactagtatagtgcccgtgcgttgcgacggcacacaaatatagtgtccgaacttggcaaagacgatgcccatgtccatgcgtcctaccagatactggagatattgtgtttcagattcactgctagaagcaatcaacatatgcaagtcttaaaaaaaaaaaaaaaaaaaaaaaaaaaaa

SEQ ID NO: 20 - protein, 157 aa, Zea mays
MYPPKASGDPAAGAAPVTGFPVGGPAASSQWSSGLLDCFDDCGLCCLTCWCPCITFGRVAEIVDRGATSCGTAGALYAVLAYFTGCQWIYSCTYRAKMRAQLGLPETPCCDCLVHFCCEPCALCQQYKELKARGFDPVLGWDRNATMLPPSAQGMGR

SEQ ID NO: 21 - DNA, 1753 bp, Zea mays
cttggatcattgacgctgcactgctgagcgcgttcaggagtaatatatataggaacgaaagttaccgagaaactggaagatcctaaactgttgccaggacatggacatcatgcatggggatctcagaaggaatggttcaaaccagagcgtgcccagtcggcatggatggatcgatatggaggccctctctcctctggcggatcaatccatgtatccatgagagagagagagagagaggcataggccaagactaggccttttgttttgagatgggctgctgctatctagagcgatactgctatatagtggtaccaagaggaccaaaggtcgcgttgcctgaaaccaccgagatcatacagaaactaatcgccgcaacgcatccaaacccgaccaactgacggcgcgcgtctgcgtgaacagctcgcagcatgccgggggagtggtccgtggggctctgcgactgcttcggggatcttcacacctgtacgcagtgcaaacaaacctgctagctatagcaagctactctcctctctctctcttcttcttcttcccgtacgtactacgatgcatgtttttcagctctccgtcccttctctcccggccgacgatcgaccacgttgcaggttgcctgacgctctggtgcccctgcgtcacgttcggccgcaccgcggagatcgtggacagaggctccacgtgtacgcgttccacttcactcccatactactactgcccccctcgtctcgtctccatctctcctgttccttttggctgacgatccgtcccgcattgcatatttgcagcgtgctgcatgagtggcacactgtactacctgctgtcgacgataggctggcagtggctgtacggctgcgccaagcgctcctccatgcggtcgcagtacagcctgcgagagtccccctgcatggactgctgcgtccacttctggtgcggcccctgcgcgctctgccaggagtacacggagctccagaaacgcggcttccacatggccaaaggtatcagctcccccccccatcttcccacagtttaactaggcggcctttgatggttccatgatcaccgtgcctctacaaaaccattatattccttttatttgcaggatgggaagggagcaacaaggtggtggggtgcttccatgggatgacgacgccaccaaggaagcaatccatgtgcttttaggatagcatagctccatcgttctatataatatgcgctttattttgaataaaaatatatgggtctgtctattgatgctttatttagagctcgttgggtctcattagttcctattgctcatagttatggtttccatttatttaaatcatcgattgttcactttattttacgttgattttatgattattacatggcgtttgagcctctgccggcctcttacgtgagacccagtaattaataaagcaatcaaaacaaggattagatatacctatagatatgcattaagtggatcagcttctaaataaacataagagcagttataatatgttttgccatagatttttgccaagttagaggagaaagaggggaagaaactaacggaccactaattatcagtcgagagattccaaaatagtgctccatccgtcacaaaatataattctttatccatttattaaccttaggatatagtttaaagttggtatgtatatctatatttattattatttattctaacgtgcatagaaaaagattattagaaagaactatgttttgggacaga

SEQ ID NO: 22 - protein, 160 aa, Zea mays
MDIMHGDLRRNGSNQSVPSRHGWIDMEALSPLADQSIMPGEWSVGLCDCFGDLHTCCLTLWCPCVTFGRTAEIVDRGSTSCCMSGTLYYLLSTIGWQWLYGCAKRSSMRSQYSLRESPCMDCCVHFWCGPCALCQEYTELQKRGFHMAKGISSPPHLPTV

SEQ ID NO: 23 - DNA, 1177 bp, Zea mays
  misc_feature 452, 453, 454, 455, 456, 457, 458, 459, 460, 461, 462,463, 464, 465: n = A,T,C or G
gcttatcggagatgtatctggcagctatgccctacgaaccgtacggggtggcggcggcgccagtcgtgtccttccccgttgccggagcggccagggcacaggcagtggtcgtcgggcctcttcgactgcttggacgagtcccgcgtctcctgtaagcctagttagcaccctcgatgttcgtcagctcgatcttgaacttttgctaggtcgtatagtaggtagctgagctcaccggcaggcacaggctgcctgacgcacttgtgcccgtgcgtcacgttcgggcgggatcgcggcgcgacgtcgtgcgcgacgggcggggcgctatacgcgctcatcgcctgcctctcggcgtcgcggtgccagtgggtgtattcctgcacgtaccgcgccgtgatgcgctcgcagttgggcctcccggaggcgccatgcgccgactgcctcgtccacctaannnnnnnnnnnnnngcgctctgccagcagtacagggagctcaaggcttggggcctcgagccctcaaccccgccatcggccgggacttgaacgatgccatgtacccgccgccggcgcaggggatgcgacggcactgatcggtggatccctccatgtatcgggtccatttcgtttgtttgttgacgacagatgcagacacagtacaaggtgtccacctacaccacacgccaggctggaccaccctccatatatatagggtgagtaaattggaagtcatatgctttcaaatattatagaaagacctgaccctttacccccgacctggctgaaacatgcatacgccaggctgtcaactgtgtcactgcccgtgcaccaggtcaaccttccccacgatcatcaggcaaacacctggtccaaaatcaacctcccgcatgggacgcaaaccataaattgtggttctgtttttacacaaatagagtaaaactgtcaacataaatagtgtaaatagttcagagagatagcataaaaaccgcgccacatgctcatgcgcaggtgcatgcatgcagattctatttttatgatagatccaataattatgacatggcgtgagaattttaattatatgtaatcaaattttagatgacattttcgtttccattgcatgcgcgcaaaaaattacatgacaggaatgtgcgagatgaaaggaaattgattgacatatatggaa

SEQ ID NO: 24 - protein, 148 aa, Zea mays
  VARIANT 120, 121, 122, 123, 124: Xaa = Any Amino Acid
AYRRCIWQLCPTNRTGWRRRQSCPSPLPERPGHRQWSSGLFDCLDESRVSCCLTHLCPCVTFGRDRGATSCATGGALYALIACLSASRCQWVYSCTYRAVMRSQLGLPEAPCADCLVHLXXXXXALCQQYRELKAWGLEPSTPPSAGT

SEQ ID NO: 25 - DNA, 813 bp, Zea mays; identical to SEQ ID NO: 3

SEQ ID NO: 26 - DNA, 546 bp, Zea mays
atgtatcccaaggcggcagacgaaggtgcgcagccgctggccacgggcatccccttcagcggcggcggcggctactaccaggcgggcggcgcgatggcggcggcgttcgcggtgcaggcgcaggcgcccgtcgccgcctggtccaccgggctctgcaactgcttcgacgactgccacaactgctgcgtgacgtgcgtgtgcccgtgcatcacgttcgggcagaccgcggagatcatcgaccggggctccacgtcctgcggcaccagcggggcgctgtacgcgctcgtcatgctgctcaccggctgtcagtgcgtctactcctgcttctaccgcgccaagatgcgcgcgcagtacggcctccaggtgagcccctgctccgactgctgcgtgcactgctgctgccagtgctgcgcgctctgccaggagtaccgcgagctcaagaagcgaggcttcgacatgagcataggatggcatgcgaacatggagaggcaggggcgcgccgccgccgccgtgccgccgcacatgcatcctgggatgacccgctga

SEQ ID NO: 27 - protein, 181 aa, Zea mays; identical to SEQ ID NO: 4

SEQ ID NO: 28 - DNA, 997 bp, Zea mays
catctcttttttgattgttacattgccaaaagtatatggagaatcatttactttgctttaaaaatagacatgcccattagcattaatcatattatcgggtcttgggaatctaataatgctctggcatacaaaacattattactgactggaatatccgctttattttggtccatctggctctgtcgaaatgaggtggtgtttaatcataaaccaataccatcaattgtgcaggttattttcagggttactcactggttcagattctggagacttctacagaaggaggaaaagcaccaacaaattctcgatgcctgtcgagctttggaggtggtggcgatggaagtttttgcaatgcatggatggcgctcgaatgcaagaattgaagatggctagtgttttctatagtcatggcattttgttttctttgagttagtttaattattcaagcaattgctctatggtccaataatcgttatgtaatggctgtacgcatttgtatgctaaagccggaactcttgtttccataatcaaaaaaaaaatagaaaagacaatacatgctgctgcgactgtgcatcggaatcgcgggatgtgcaaatatactaatgctctcgtgtttagtgcagtaaccaaacccttccttgatttgatattaaggaactgcatctgcatcggattcccgttgaaaagtaacgcggcattatttattggtatggtcacctaaaatactattttataatatagaccgtattatttatataatagattttgaaatagataatggagataattaagctgttgaagatagcctttaactagttagtaccgcagtacgtgagtggtgacttgagggagacctctcgagtcgagtcttgccgacctggggcctgctgcgcctggatgaagagctgtgctgagagaatcattaaagcagcaccgggtccttgctttgcgcgttgctgttcgcataaacaagagatcctagttctactcccaagca

SEQ ID NO: 29 - DNA, 997 bp, Zea mays
tctccgcgtggagttttatagcgttgttcttttttagcaccgacatgacatattacatagaatgaaatgaaagttaaatggaatttatgggaagttttctgtggagatttaattcgcatgaatgacttattaagagttgataaatatgccaagcagagagtttgatagccataaagctcacttcttctcttttctaggataattgtcacgtacgtcatccaaaatatttaaaactttttgagtacaaataaaactccaacaagatgagattagtcttatcacacgttccactgacacttggaataatataatgtggatggacgccttatgtcattagtgggaagaatagacaacaaccgaacaacgtgccattatattggcgaaagcttgactagcggattagtttgtatcggtggtcctgttagtctttgggcccgcgggaaagcgagccgtgccatttctacgcgcgcactttattttatttattgtttacttcggtcgtcgatgttgggcagggataagtcttgcttccggtccacggacgcatgctacgttaccatctctagctagctagctaggcggacacttgacgtcgaaacaagggcagaaataaaacaacgtgatcattccatggcagtgcggcaggccacatacatgcatgaggatgagcatcgcgagtcgcggcgctccacgtaacccttgtaccaccatccggtccgggtgtggccagccgcgtcgtactcggcggccgggagacgactttgacctgcttgcacggctggcccggggccatcgtcagttcgtcacaacctgacgtagtgcttgggcacagcgcactgaaaaaaaaaacattaccgtcgcgccgagataaatacgcgctgcggccggacaggctagctagaccggacgcgcacaaggcaataaataaaaagcccaacgctgggctccgctagctctcggtctctctggagtctggactcgcctccacggctccacc

SEQ ID NO: 30 - DNA, 1000 bp, Zea mays
cttcgtctagcgctaggtcttctctttcgactctgatctttgccgtcgtcactcggttctctagctcttgaagcgtgcacctacaagtcgcccactctccagctctctccactcttcggcaagggtgagactgaggcgggtcctgtgtcgtgcatgttgtgcggtgcgccctgctggccagcccggctgcccgacctgagctagcactacgcagctgcttcactgactttggtgcttgcagcttccttgtgccttgtgtggcccatgaccgtgtgaccatccttccagcgtagcctaatcaacctcatacattcactgattgtgagcactacgacactatatgccacaataagcaacctcaacattcattcatgttgtgctactccgaacctattagcctattcatcacacaatacaatttcattatacaaataagtgaagcggtgaaagcagaggcgcctggtacagaaacttttttgcctttttagaccacgcaccggcgaggaagcaatcaattagaaaccccacgagcgaagtttgacataggcaaacaccaaaaagacatctctgtcactgagaactgcaagttcaaagtcatgtaaaaaaccggtcataccttaaattttactcgtcctccgcaactcccaatatattgtacaacgggtaattacggaacaataactaccttacaatattaatgattgaaaaaaaagaaagcataaatgataaaattcgtatgttaagcgtccattcatttttactaacgggtcacactattactccaatcatcgtacattaatcggcggccgtcacccatatattcaacattcgtttcgctgttgtgttgggccttactacatttgtagacggcatctagggcctcttttgacctttctagatgtcatctataaatacgcatacttggcaagctgatgcatcaactcatcacccgtagtatcaattatcataacctccgactccgacgatctgaactgtgaag

SEQ ID NO: 31 - DNA, 442 bp, Zea mays
gttatattttgtctaaaattaatcgtccgaattaaaaaactaggtggatatttgtttgaggatgaagtggtgcatcatgagctcatttctcataaatttggtgggattctatttatcatattaatacaaactaattatgaggtgttgataaatcatcttattgtattccataaatcaaataaaaagaagtaaggagtgagaagataatagactaggttatttatcaaaccaaatactttataactttagttaaaaaagttaggtcaagtataagcaccaaacaaaagagccgaggaggatgtttaacttgggcctgatcagggcccggcgtgcttcatgtaagctccggcccgatcgatgagaggagtcagacggaggcaaggccggggtcggcagcagaggggagcagagagatctcattctccgatccgcccaggccacc

SEQ ID NO: 32 - DNA, 815 bp, Zea mays
tgggaattacttaggagctgtgggagtagcttgcattgttgcaaaaaaggactataatattgtgtgcgtgtctttggttatgaataaaacaaaatagataatttgccatgtctcttcctatctcgtttgcattattttggggaaaaaaaggaccgtaataatgcgtgtgtctttggctgtatgcatgaatacaaaatagacaaaataatctctccagagtggaacgctaagaaataatggaagagacaggagggacaactaacgactgttaggttaggtagtatttgaatctgaagtatagtgggatggagcatatctcggattggatagagtgactttaaaaaggaaaattcagttccaatcatccagctctctaaaatctctcggatgaaaccgctggcatggagctacttcatcccgatcaccaaaccctacgttactcaagtgacaagtggtgggaggccaaacgctagctgggccccaccctagtgcatccgccaagagcacatgcatgcccgtgccgtgcgcggtgagccccgccgccgagttggctgcagagctgcggagtcttggcgaagaccccgcgctgggcgtcgccgctgctgagtcggtcaacgaggcgtaaccagcgagccagcgacgcgcctttgggttgggattggacccggtcgtcccatcccagccccgcctatgtaaagagcacccgccgctgcccagctctctcattcctgtcggcaaggtgaacgcgagagccgaccgtctcagtgtgcgcgcgaagaagagaaggaggaggaggaggaggaagaagaagaaga

SEQ ID NO: 33 - DNA, 630 bp, Zea mays
gggtgtgtgttttggtaaaatatgaacgaaatgaacctcgctcaagtctcgaacgttgggccctagccccaacctgtttccgactgctgttttaactactgcagttataattcctgttccgactgctgttttaactattacagttataatttatagagacaaaaataaaaatactatcagtccgaagctgatacatattaaagtcaagtcctagctcttgggtgcgtgtgtgctgctcaacaccccaacgcgtggaacgggaaagccggaaaggcccccgcccgcacacgccgccgccgctgccaattcggtcaacgtacgataccagccgcgggccggcgagtccaggcggtcgcagtcgcacgtcgcgacgcgccttagcattggaccgagccgcggccacttgctttgcaattgcatttgccgggatccatgccagcagctccgtcctatatgtgaagcacccggccgcaaggctctctcgctcattcacggcaaattaagcgagcgccggccgaggcagacacaacccgcagcaacaacttgcatcggcacggacgaccgattgagagcctcggcgttcgtacgccacgccaccaagagactctgtgcaacaagtgaagggacgaa

SEQ ID NO: 34 - DNA, 100 bp, Zea mays
cttggatcattgacgctgcactgctgagcgcgttcaggagtaatatatataggaacgaaagttaccgagaaactggaagatcctaaactgttgccaggac

SEQ ID NO: 35 - DNA, 89 bp, Zea mays
gcttatcggagatgtatctggcagctatgccctacgaaccgtacggggtggcggcggcgccagtcgtgtccttccccgttgccggagcg

SEQ ID NO: 36 - DNA, 17 bp, Artificial Sequence
  Oligonucleotide
gatcggctgggaggcta

SEQ ID NO: 37 - DNA, 17 bp, Artificial Sequence
  Oligonucleotide
gatctgtatgcggtatc

SEQ ID NO: 38 - DNA, 17 bp, Artificial Sequence
  Oligonucleotide
gatccagacggtacata

SEQ ID NO: 39 - DNA, 17 bp, Artificial Sequence
  Oligonucleotide
gatctttaactgatatg

SEQ ID NO: 40 - DNA, 17 bp, Artificial Sequence
  Oligonucleotide
gatctttcaacaacaaa

SEQ ID NO: 41 - DNA, 17 bp, Artificial Sequence
  Oligonucleotide
gatcatcgcgtacgtac

SEQ ID NO: 42 - DNA, 17 bp, Artificial Sequence
  Oligonucleotide
gatctggtcgaggacga

SEQ ID NO: 43 - DNA, 17 bp, Artificial Sequence
  Oligonucleotide
gatctactcgtgcacgt

SEQ ID NO: 44 - protein, 126 aa, Lycopersicon sp.
  VARIANT 34: Xaa = Any Amino Acid
SCHFIMSMHDSIPGCLTCWCPCITFGRVPEIVDXGATSCGTAGALYPVLAYFPGCQWIYSCTYRAKMRAQLGLPETPCCDCLVHFCCEPCALCQQYKELKARGFDPVLGWDRNATMLPPSAQGMGR

SEQ ID NO: 45 - protein, 163 aa, Lycopersicon sp.
MYQTVGYNPGPMKQPYVPPHYVSAPGTTTARWSTGLCHCFDDPANCLVTSVCPCITFGQISEILNKGTTSCGSRGALYCLLGLTGLPSLYSCFYRSKMRGQYDLEEAPCVDCLVHVFCEPCALCQEYRELKNRGFDMGIGWQANMDRQSRGVTMPPYHAGMTR